(19) United States
(12) Patent Application Publication
(10) Pub. No.: US 2021/0059590 A1
(43) Pub. Date: Mar. 4, 2021

(54) IDENTIFICATION OF AND TREATMENT FOR PERSONS AT HIGH RISK FOR DEVELOPING MAJOR DEPRESSIVE DISORDER

(71) Applicant: The Trustees of Columbia University in the City of New York, New York, NY (US)

(72) Inventors: Mate Istvan Milak, New York, NY (US); J. John Mann, Bronx, NY (US)

(21) Appl. No.: 16/961,769

(22) PCT Filed: Jan. 14, 2019
(86) PCT No.: PCT/US2019/013448

§ 371 Date: Jul. 13, 2020

Related U.S. Application Data
(60) Provisional application No. 62/616,505, filed on Jan. 12, 2018.

(51) Int. Cl. A61B 5/16; A61K 33/00; A61K 31/135; A61K 45/06; A61P 25/24; A61B 5/00; A61B 6/03; A61B 6/00

(52) U.S. Cl. CPC A61B 5/165; A61K 33/00; A61K 31/135; A61B 6/501; A61P 25/24; A61B 5/7275; A61B 6/037; A61K 45/06

(57) ABSTRACT

This invention relates to the field of treatment, prevention, and identification of persons at high risk of developing major depressive disorder based upon elevated concentration of serotonin 1A receptors (i.e., elevated serotonin 1A receptor binding potential). In particular, the current invention identifies offspring of parents with major depressive disorder who are at significantly elevated risk of developing major depressive disorder before any symptoms have occurred and treats these patients prior to any symptoms and more importantly before developing major depressive disorder. The invention also relates to the identification of persons who have discontinued treatment for major depressive disorder based upon elevated concentration of serotonin 1A receptors (i.e., elevated serotonin 1A receptor binding potential).

### CROSS REFERENCE TO RELATED APPLICATION

The present application claims priority to U.S. Patent Application Ser. No. 62/616,505 filed Jan. 12, 2018, which is hereby incorporated by reference in its entirety.

This invention was made with government support under MH074813, MH062185 and MH040695, awarded by NIH. The government has certain rights in the invention.

### FIELD OF THE INVENTION

This invention relates to the field of treatment and identification of persons at high risk of developing major depressive disorder based upon elevated concentration of serotonin 1A receptors (i.e., elevated serotonin 1A receptor binding potential). In particular, the current invention identifies offspring of parents with major depressive disorder who are at significantly elevated risk of developing major depressive disorder before any symptoms have occurred allowing treatment before they become symptomatic.

The invention also relates to the identification of persons who have discontinued treatment for major depressive disorder based upon elevated concentration of serotonin 1A receptors (i.e., elevated serotonin 1A receptor binding potential).

### BACKGROUND OF THE INVENTION

Depression is the leading cause of disability worldwide, affecting over 300 million people (WHO, 2017). Over sixteen million adults in the United States are afflicted with major depressive disorder (MDD) (NIMH, 2015). In 2010, the economic burden caused by MDD in the United States alone was estimated to be $210.5 billion, an increase from that in 2005 of 21.5% (Greenberg et al., 2015). Prevention and pre-intervention research programs addressing this major public health burden are underrepresented in the National Institutes of Health portfolio.

Depressive illnesses are estimated to be 40% heritable (Uhl and Grow, 2004), and offspring of individuals with early-onset depression are at higher risk of developing these disorders (Mann et al., 2005). The serotonin (5-HT) system has been implicated in major depressive disorder (MDD) (Blier et al., 1990), and offspring of MDD patients report transient depression after serotonin depletion by acute tryptophan depletion (Klaassen et al., 1999).

The best way to stop familial transmission of MDD is to target individuals at high risk for developing MDD for preventive interventions. One in about four individuals with a parent with MDD is at risk of developing MDD. To intervene with a treatment to prevent the onset of an illness like MDD in someone who is at high risk and still asymptomatic, without exposing those who are not truly susceptible to developing MDD, is to have a biological test, that can detect the endophenotype that is present in those truly at risk, before any symptoms appear. This biological test can be used to screen and identify those who carry an endophenotype for depression and are truly at risk (carrier high-risk or CHR). This test will be negative in those who are not susceptible and not at a heightened risk (non-carrier high risk, or NCHR, individuals). Being able to deliver preventive interventions to the CHR individuals who are most likely to develop MDD may help reduce the worldwide prevalence of MDD and associated economic burden. As of now there is no biological test to identify correctly the offspring or relatives of MDD probands at higher risk.

Additionally, the 5-HT_{1A} autoreceptor is a major target of SSRI antidepressant action. It has been previously reported by the inventors that selective serotonin reuptake inhibitor (SSRI) treatment of a major depressive episode (mean duration=7 weeks) in individuals with MDD produced an 18% reduction in autoreceptor binding (Gray et al., 2013). However, it was not known how long this effect persisted following medication discontinuation. Since SSRIs are thought to work by reversing the MDD-related elevation in autoreceptor binding once this effect wears off, the patient may no longer be protected from a return of symptoms or recurrence of the episode or relapse into a new episode. As of now there was no biological test to identify when a patient was no longer protected by the action of SSRIs or to identify when a patient may have ceased treatment but has not been forthcoming about such.

### SUMMARY OF THE INVENTION

The current invention is based on the discovery that a particular pattern of higher serotonin 1A (5-HT_{1A}) receptor concentration in the brain is a biologic marker and indeed an endophenotype for familial transmission of major depressive disorder (MDD). It is shown herein that offspring of parents with MDD, who actually develop MDD have the pattern of higher serotonin 1A receptor concentration before becoming symptomatic with MDD, and offspring who do not develop MDD do not have the pattern. Thus, using this endophenotype, offspring of parents with MDD can be identified as having significantly elevated risk for developing MDD, and treated, even before symptoms occur.

Thus, one embodiment of the present invention is a method of treating and/or preventing a subject for major depressive disorder, comprising: detecting an elevated, increased or higher concentration of serotonin 1A receptors or elevated serotonin 1A receptor binding or related binding index in the brain of the subject; and administering to the subject a therapeutically effective amount of therapeutic agent for major depressive disorder and/or treating the subject with psychotherapy to reduce the degree of this biological abnormality or endophenotype so that the brain pattern shifts from a high risk pattern, i.e., elevated, increased or higher concentration of serotonin 1A receptors or an elevated serotonin 1A receptor binding in the brain, to a lower risk pattern.

In some embodiments, the subject has at least one parent with major depressive disorder.

In some embodiments, the subject is asymptomatic of MDD.

In some embodiments, the subject is between the ages of 18 and 32 years. In some embodiments, the subject is under 18 years old.

In some embodiments, the entire brain is scanned. In some embodiments, one or more areas of the brain are scanned including: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyms; temporal lobe; and raphe nuclei.

In some embodiments, the detection of the elevated, increased or higher concentration of serotonin 1A receptors or elevated serotonin 1A receptor binding potential is performed by the use of a scan of the brain including but not limited to positron emission tomography (PET), and single-photon emission computed tomography (SPECT).

In some embodiments, the scan is performed more than once.

In some embodiments, the concentration of serotonin 1A receptors or serotonin 1A receptor binding potential is compared to a reference value. In some embodiments, the reference value is the concentration of serotonin 1A receptors or the serotonin 1A receptor binding potential of a healthy comparison subject or group.

In some embodiments, the reference value of the offspring who developed MDD.

In some embodiments, the reference value is the serotonin 1A receptor binding potential binding potential (BP_{F}) that is equal or greater than about 80 ml/cm³.

In some embodiments, the reference value is the serotonin 1A receptor binding potential binding potential (BP_{F}) that is equal or greater than about 70 ml/cm³.

In some embodiments, the reference value is the serotonin 1A receptor binding potential binding potential (BP_{F}) that is equal or greater than about 60 ml/cm³.

In some embodiments, the reference value is the serotonin 1A receptor binding potential binding potential (BP_{F}) that is equal or greater than about 50 ml/cm³.

In some embodiments, the area of the brain from which the serotonin 1A receptor binding potential is detected and compared is the hippocampus.

In some embodiments, the area of the brain from which the serotonin 1A receptor binding potential is detected and compared is the parahippocampal gyms.

In some embodiments, the area of the brain from which the serotonin 1A receptor binding potential is detected and compared is the insula.

In some embodiments, the area of the brain from which the serotonin 1A receptor binding potential is detected and compared is the temporal lobe.

In some embodiments, the area of the brain from which the serotonin 1A receptor binding potential is detected and compared is the amygdala.

In some embodiments, the area of the brain from which the serotonin 1A receptor binding potential is detected and compared is the anterior cingulate.

In some embodiments, the area of the brain from which the serotonin 1A receptor binding potential is detected and compared is the medial prefrontal cortex.

In some embodiments, the area of the brain from which the serotonin 1A receptor binding potential is detected and compared is the dorsolateral prefrontal cortex.

In some embodiments, the area of the brain from which the serotonin 1A receptor binding potential is detected and compared is the cingulate body.

In some embodiments, the area of the brain from which the serotonin 1A receptor binding potential is detected and compared is the orbital prefrontal cortex.

In some embodiments, the area of the brain from which the serotonin 1A receptor binding potential is detected and compared is the raphe nuclei.

In some embodiments, the area of the brain from which the serotonin 1A receptor binding potential is detected and compared is the parietal lobe.

In some embodiments, the area of the brain from which the serotonin 1A receptor binding potential is detected and compared is the occipital lobe.

In some embodiments, multivoxel pattern analysis is used to analyze the data in the scans. In some embodiments, using MVPA the reference value is an AUC of about 0.80.

In some embodiments, the percent free fraction (f_{P}) of a PET tracer is used in the calculation of BP_{F}. In some embodiments, the reference value is equal or less than about 6.2% free fraction (f_{P}).

In some embodiments, the therapeutic agent includes but is not limited to selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine and monoamine oxidase inhibitors (MAOIs).

In some embodiments, the psychotherapy is selected from the group consisting of Interpersonal Therapy and Cognitive Behavioral Therapy.

A further embodiment of the present invention is a method of identifying a subject who is at high risk for major depressive disorder or has major depressive disorder, comprising: detecting an elevated, increased or higher concentration of serotonin 1A receptors or an elevated serotonin 1A receptor binding or related binding index in the brain of the subject.

In some embodiments, the subject has at least one parent with major depressive disorder.

In some embodiments, the subject is asymptomatic of MDD.

In some embodiments, the subject is between the ages of 18 and 32 years. In some embodiments, the subject is under 18 years old.

In some embodiments, the entire brain is scanned. In some embodiments, one or more areas of the brain are scanned including: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyms; temporal lobe; and raphe nuclei.

In some embodiments, the detection of the elevated, increased or higher concentration of serotonin 1A receptors or elevated serotonin 1A receptor binding potential 1 is performed by the use of a scan of the brain including but not limited to positron emission tomography (PET), and single-photon emission computed tomography (SPECT).

In some embodiments, the scan is performed more than once.

In some embodiments, the concentration of serotonin 1A receptors or serotonin 1A receptor binding potential is compared to a reference value. In some embodiments, the reference value is the concentration of serotonin 1A receptors or the serotonin 1A receptor binding potential of a healthy comparison subject or group.

In some embodiments, the reference value of the offspring who developed MDD.

In some embodiments, the reference value is the serotonin 1A receptor binding potential binding potential (BP_{F}) that is equal or greater than about 80 ml/cm³.

In some embodiments, the reference value is the serotonin 1A receptor binding potential binding potential (BP_{F}) that is equal or greater than about 70 ml/cm³.

In some embodiments, the reference value is the serotonin 1A receptor binding potential binding potential (BP_{F}) that is equal or greater than about 60 ml/cm³.

In some embodiments, the reference value is the serotonin 1A receptor binding potential binding potential (BP_{F}) that is equal or greater than about 50 ml/cm³.

In some embodiments, the area of the brain from which the serotonin 1A receptor binding potential is detected and compared is the hippocampus.

In some embodiments, the area of the brain from which the serotonin 1A receptor binding potential is detected and compared is the parahippocampal gyms.

In some embodiments, the area of the brain from which the serotonin 1A receptor binding potential is detected and compared is the insula.

In some embodiments, the area of the brain from which the serotonin 1A receptor binding potential is detected and compared is the temporal lobe.

In some embodiments, the area of the brain from which the serotonin 1A receptor binding potential is detected and compared is the amygdala.

In some embodiments, the area of the brain from which the serotonin 1A receptor binding potential is detected and compared is the anterior cingulate.

In some embodiments, the area of the brain from which the serotonin 1A receptor binding potential is detected and compared is the medial prefrontal cortex.

In some embodiments, the area of the brain from which the serotonin 1A receptor binding potential is detected and compared is the dorsolateral prefrontal cortex.

In some embodiments, the area of the brain from which the serotonin 1A receptor binding potential is detected and compared is the cingulate body.

In some embodiments, the area of the brain from which the serotonin 1A receptor binding potential is detected and compared is the orbital prefrontal cortex.

In some embodiments, the area of the brain from which the serotonin 1A receptor binding potential is detected and compared is the raphe nuclei.

In some embodiments, the area of the brain from which the serotonin 1A receptor binding potential is detected and compared is the parietal lobe.

In some embodiments, the area of the brain from which the serotonin 1A receptor binding potential is detected and compared is the occipital lobe.

In some embodiments, the percent free fraction (f_{P}) of a PET tracer is used in the calculation of BP_{F}. In some embodiments, the reference value is equal or less than about 6.2% free fraction (f_{P}).

A further embodiment of the present invention is a method of treating and/or preventing a subject for major depressive disorder, comprising: detecting a serotonin 1A receptor binding potential pattern in the brain of the subject the same or similar to a subject with MDD; and administering to the subject a therapeutically effective amount of therapeutic agent for major depressive disorder and/or treating the subject with psychotherapy.

In some embodiments, the subject has at least one parent with major depressive disorder.

In some embodiments, the subject is asymptomatic of MDD.

In some embodiments, the subject is between the ages of 18 and 32 years. In some embodiments, the subject is under 18 years old.

In some embodiments, the entire brain is scanned. In some embodiments, one or more areas of the brain are scanned including: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyms; temporal lobe; and raphe nuclei.

In some embodiments, the detection of the serotonin 1A receptor binding potential is performed by the use of a scan of the brain including but not limited to positron emission tomography (PET), and single-photon emission computed tomography (SPECT).

In some embodiments, the scan is performed more than once.

In some embodiments, multivoxel pattern analysis is used to analyze the data in the scans.

In some embodiments, the therapeutic agent includes but is not limited to selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine and monoamine oxidase inhibitors (MAOIs).

In some embodiments, the psychotherapy is selected from the group consisting of Interpersonal Therapy and Cognitive Behavioral Therapy.

A further embodiment of the present invention is a method of identifying a subject who is at high risk for major depressive disorder or has major depressive disorder, comprising: detecting a serotonin 1A receptor binding potential pattern in the brain of the subject the same or similar to a subject with MDD.

In some embodiments, the subject has at least one parent with major depressive disorder.

In some embodiments, the subject is asymptomatic of MDD.

In some embodiments, the subject is between the ages of 18 and 32 years. In some embodiments, the subject is under 18 years old.

In some embodiments, the entire brain is scanned. In some embodiments, one or more areas of the brain are scanned including: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyms; temporal lobe; and raphe nuclei.

In some embodiments, the detection of the serotonin 1A receptor binding potential is performed by the use of a scan of the brain including but not limited to positron emission tomography (PET), and single-photon emission computed tomography (SPECT).

In some embodiments, the scan is performed more than once.

In some embodiments, multivoxel pattern analysis is used to analyze the data in the scans.

Further embodiments of the invention are based on the discovery that any antidepressant-associated downregulation of 5-HT_{1A} autoreceptor binding reversed within two weeks of medication discontinuation.

Thus, a further embodiment of the present invention is a method of identifying a subject with major depressive disorder who has discontinued treatment for the major depressive disorder, comprising: detecting an elevated, increased or higher concentration of serotonin 1A receptors or an elevated serotonin 1A receptor binding potential or related binding index in the brain of the subject.

In some embodiments, the subject is between the ages of 18 and 32 years. In some embodiments, the subject is under 18 years old.

In some embodiments, the subject has discontinued treatment for at least two weeks.

In some embodiments, the entire brain is scanned. In some embodiments, one or more areas of the brain are scanned including: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyms; temporal lobe; and raphe nuclei.

In some embodiments, the detection of the elevated, increased or higher concentration of serotonin 1A receptors or elevated serotonin 1A receptor binding potential 1 is performed by the use of a scan of the brain including but not limited to positron emission tomography (PET), and single-photon emission computed tomography (SPECT).

In some embodiments, the scan is performed more than once.

In some embodiments, the concentration of serotonin 1A receptors or serotonin 1A receptor binding potential is compared to a reference value. In some embodiments, the reference value is the concentration of serotonin 1A receptors or the serotonin 1A receptor binding potential of a healthy comparison subject or group.

In some embodiments, the reference value is the serotonin 1A receptor binding potential binding potential (BP_{F}) that is equal or greater than about 80 ml/cm³.

In some embodiments, the area of the brain from which the serotonin 1A receptor binding potential is detected and compared is the hippocampus.

In some embodiments, the reference value is the serotonin 1A receptor binding potential binding potential (BP_{F}) that is equal or greater than about 40 ml/cm³.

In some embodiments, the area of the brain from which the serotonin 1A receptor binding potential is detected and compared is the raphe nuclei.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1A is a graph showing that High Risk (HR) subjects differ significantly from Healthy Volunteers (HVs) (p=0.004) in 5-HT_{1A} binding potential (BP_{F}) but do not differ from Depressed Not-Recently Medicated (NRM) Major Depressive Disorder (MDD) subjects (p=0.884) across all 13 regions of interest (anterior cingulate=ACN; amygdala=AMY; cingulate body=CIN; dorsolateral prefrontal cortex=DOR; hippocampus=HIP; insula=INS; medial prefrontal cortex=MED; occipital lobe=OCC; orbital prefrontal cortex=ORB; parietal lobe=PAR; parahippocampal gyrus=PIP; temporal lobe=TEM; raphe nuclei=RN). The bars of the graph represent the group mean 5-HT_{1A} binding potential (BP_{F}) weighted by bootstrap standard errors for each region, while the error bars display the corresponding weighted standard deviations.

FIG. 1B are representative images of PET scans that show the average [¹¹C]WAY100635 5-HT_{1A} receptor BP_{F} of nine high risk subjects and 51 healthy volunteers. Binding potential maps, corrected for free fraction, were derived from the PET scans of all subjects. Abbreviations: HIP=hippocampus, AMY=amygdala.

FIG. 2A shows voxel-based 5-HT_{1A} BP_{F} contrast maps for group differences across Healthy Volunteer (HV), Depressed Not-Recently Medicated (NRM) Major Depressive Disorder (MDD) and High Risk (HR) subjects (omnibus F-test using family-wise error rate (FWE) correction at p<0.05) on the left-hand side. Plots on the right-hand side show the average (mean-centered) BP_{F} values adjusted for sex in a midbrain cluster in the vicinity of dorsal raphe nucleus (top row), left inferior temporal gyms (middle row) and ventromedial prefrontal cortex (PFC, bottom row). Statistical maps were thresholded using FDR correction at p<0.05, cluster size (k)>10. Error bars represent 90% confidence intervals (CI). Table 3 lists all regions surviving p<0.05 FWE correction.

FIG. 2B is a graph of BP_{F} in High Risk (HR) subjects versus an age-matched subgroup of Healthy Volunteers (HV) (N=9, p=0.03). HR subjects have significantly higher BP_{F}.

FIG. 3 shows the results of MVPA using whole-brain 5-HT_{1A} BP_{F} maps to classify High Risk (HR), Depressed Not-Recently Medicated (NRM) Major Depressive Disorder (MDD) and Healthy Volunteer (HV) subjects. FIG. 3A is a graph where classification performance (AUC) was plotted versus number of features that have been ranked by their absolute t-score (in the training data). FIG. 3B shows the top 75 features (voxels) over the entire dataset that were used to train a classification model and their SVM weights are displayed neuroanatomically. Informative voxels for HR vs. HV include fusiform and parahippocampal gyrus (z=−12), ventrolateral prefrontal cortex (z=0), anterior cingulate (z=12), inferior and superior parietal lobe (z=36, 48, 60). Solid gray line represents mean for the null distribution and error bars represent 95% confidence intervals (CI) for the null distribution. Note that all HR vs. HV values are outside of the 95% CI.

FIG. 4 shows a graph of High Risk (HR) subjects who converted to major depressive disorder (MDD) show higher 5-HT_{1A} BP_{F} systematically across regions of interest compared with HR subjects who did not convert to MDD (hippocampus=HIP; parahippocampal gyrus=PIP; insula=INS; temporal lobe=TEM; amygdala=AMY; anterior cingulate=ACN; medial prefrontal cortex=MED; dorsolateral prefrontal cortex=DOR; cingulate body=CIN; orbital prefrontal cortex=ORB; raphe nuclei=RN; parietal lobe=PAR; occipital lobe=OCC).

FIG. 5 shows a graph of 5-HT_{1A} binding measured by [¹¹C]WAY-100635 BP_{F} in the brain of male and female subjects who were exposed to antidepressants, then discontinued, and antidepressant naïve (i.e., those who were never exposed to antidepressants). BP_{F} did not differ between the two groups, after accounting for age and sex. FIG. 5A shows the results in the raphe nuclei. FIG. 5B shows the results in the hippocampus.

FIG. 6 shows a graph of 5-HT_{1A} binding measured by [¹¹C]WAY-100635 BP_{F} in the hippocampus of male and female subject who were exposed to antidepressants and then discontinued. X-axis shows the weeks off antidepressant medication. There is no effect of time off antidepressants in antidepressant-exposed participants.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “subject” as used in this application means a human or a person.

The term “detection”, “detect”, “detecting” and the like as used herein means as used herein means to discover the presence or existence of.

The terms “diagnosis”, “diagnose”, diagnosing” and the like as used herein means to determine what physical disease or illness a subject or patient has, in this case a major depressive disorder (MDD).

The terms “identification”, “identify”, “identifying” and the like as used herein means to recognize a disease state or a clinical manifestation or severity of a disease state in a subject or patient. The term also is used in relation to test agents and their ability to have a particular action or efficacy.

The term “reference value” as used herein means the concentration of serotonin 1A receptors or the serotonin 1A receptor binding potential from a healthy comparison subject or healthy comparison group. As used herein the reference value can also be the concentration of serotonin 1A receptors or the serotonin 1A receptor binding potential of the offspring who developed MDD.

The term “healthy comparison subject” as used herein means a human subject who is not suffering from MDD, has never suffered from MDD, and has no relatives with MDD. A “healthy comparison group” is a group of healthy subjects who are not suffering from MDD, have never suffered from MDD, and have no relatives with MDD.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The phrase “in need thereof” indicates a subject has major depressive disorder, even if asymptomatic, is suspected of having major depressive disorder, or has a high risk of developing major depressive disorder.

As used herein, “Receptor Binding Potential”, “Binding Potential” or “BP_{F}” refers to the concentration of receptors available for binding, i.e., the ratio at equilibrium of the concentration of specifically bound radioligand in tissue to the concentration of free radioligand in tissue. Binding potential in a region of interest refers to the ratio at equilibrium of the concentration of specifically bound radioligand in tissue of the region of interest to the concentration of free radioligand in tissue (Innis et al., 2007).

### Abbreviations

HR— High Risk
HV— Healthy Volunteer
NRM—Depressed Not-Recently Medicated
MDD—Major Depressive Disorder
ROI— region of interest

An Endophenotype that Identifies Persons at Elevated Risk for Major Depressive Disorder (MDD)—Serotonin-1A (5-HT_{1A}) Receptor Binding Potential (BP_{F})

Offspring of one or both parents with MDD have about a 25-28% chance of inheriting MDD. Thus, about three quarters of these offspring will not develop the disorder. However, in those that will, it is important to identify them and treat them early even before their symptoms occur. Once a subject develops symptoms of MDD, detrimental changes occur in their brain, making treatment and therapy more difficult. The current invention of identifying those offspring of parents with MDD who will actually develop MDD before symptoms occur will allow treatment and preventive intervention of these subjects before they become too ill to respond.

An endophenotype would help to identify persons at elevated risk of developing MDD (HR, or high risk but healthy (no history of any mental disorder), young (age 18 to 32 years) offspring of MDD probands), before they become symptomatic, thereby guiding delivery of treatment and preventive interventions. A biomarker or endophenotype could also: (a) improve diagnostic classification by identification of biologic subtypes that are transmitted in families; (b) improve treatment outcome if biologic subtypes respond to different treatments; (c) guide the search for genetic and environmental factors that mediate the vulnerability to mood disorders and are modifiable to inform a prevention strategy; and (d) help develop and validate animal models for depression.

Shown herein are the novel findings of a biologic marker namely elevated serotonin-1A (5-HT_{1A}) receptor binding potential (BP_{F}) being an endophenotype, for familial transmission of MDD. In particular, it is shown herein that only some offspring of parents with MDD develop MDD and these offspring have elevated serotonin-1A (5-HT_{1A}) receptor binding potential even greater than the offspring who do not go on to develop MDD.

The operational requirements (Peterson and Weissman, 2011; Gottesman and Shields, 1973) for a biologic marker to be an endophenotype are that it: (a) is associated with the illness in the general population; (b) is heritable; (c) is a trait (i.e., present in an affected individual whether or not the illness is active); (d) co-segregates with illness within the affected families; and (e) is found in non-affected family members at a higher rate than in the general population (Leboyer et al., 1998).

Previous findings by the inventors have shown conditions (a) and (c) by demonstrating elevated 5-HT_{1A} binding in MDD during a major depressive episode (MDE) across thirteen brain regions known to have high 5-HT_{1A} receptor density (Parsey et al., 2010a; Parsey et al., 2006) and between MDEs in remitted, unmedicated MDD (Miller et al., 2009).

The work reported herein while showing criterion (e) by finding elevated 5-HT_{1A} binding in healthy offspring of MDD probands, albeit lower than those who went on to develop MDD, and more importantly shows criterion (c), by demonstrating the presence of this biomarker in those HR subjects who have later gone on to develop MDD. In other words, the subjects who later developed MDD always had the trait of elevated BP_{F}.

Previous findings did not show that the use of elevated 5-HT_{1A} binding to identify offspring of parents with MDD who will go on to develop MDD and to use therapeutic intervention to prevent and treat MDD in these asymptomatic offspring. The importance of this finding is that these offspring can be identified before they develop MDD which allows for more successful therapeutic intervention.

In this study, the 5-HT_{1A} receptor BP_{F} in healthy high risk (HR) subjects was compared to that in healthy volunteers (HV), depressed not-recently medicated (NRM) MDD and remitted NRM MDD subjects. Supervised machine learning analyses of the imaging data was utilized seeking classification of HR individuals into either MDD or HV based on differences between depressed NRM MDD subjects versus HV and remitted NRM MDD subjects vs. HV. Clinical follow-up of a subgroup of HR subjects allowed a preliminary comparison of BP_{F} between those who did and did not subsequently develop MDD (converters vs. non-converters or resilient HR subjects). This follow-up data showed two out of the five subjects who were reachable for a diagnostic interview had gone on to develop full-blown clinical depression, were diagnosed and treated (successfully) with antidepressants. At the time of the follow-up interview, both of these converters were in full sustained remission. A MVPA model trained to discriminate HV versus depressed NRM MDD subjects classified both converters as MDD, while one of three non-converters were classified as HV. This is consistent with the hypothesis that the pattern of elevated BP_{F} is an endophenotype for MDD and that MVPA of BP_{F} patterns and elevated BP_{F} can be used to predict risk for development of MDD in HR subjects.

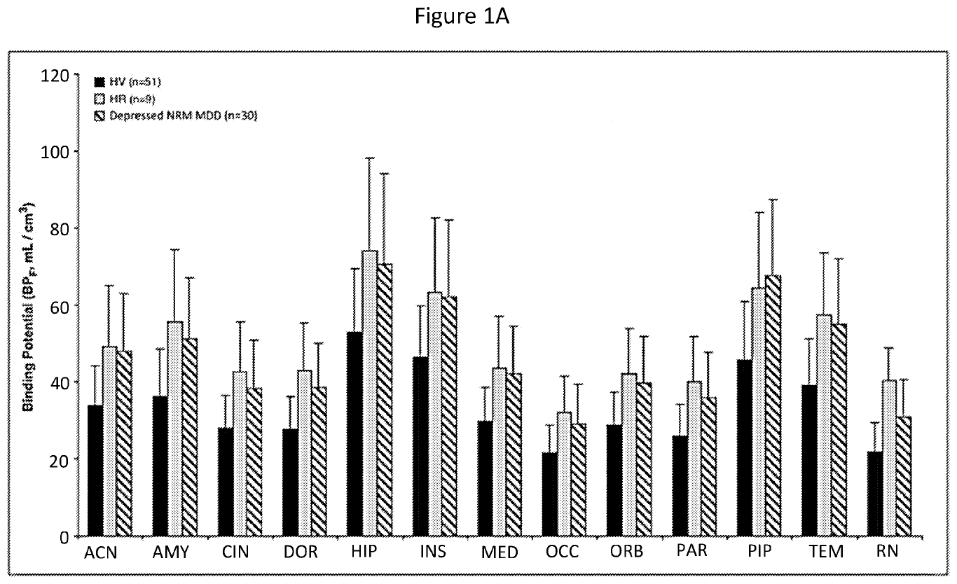

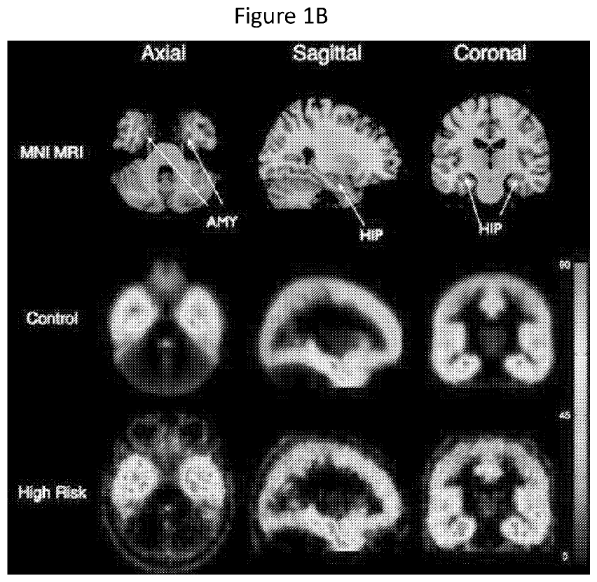

The age difference between groups, as well as a difference in injected mass between groups (Table 1), did not explain the findings (i.e., neither age nor injected mass was correlated with BP_{F}), and group differences remained when including age, injected mass, and/or dose as nuisance covariates in the model. In the current cohort, a higher binding in HR vs. HV was found whether BP_{F} (FIG. 1) or BP_{P} (p<0.001) was used, the latter of which does not require measurement of f_{P} (B_{P}=f_{P}×Bavail/K_{D}) (Innis et al., 2007). It is unlikely that these findings are an artifact of a partial volume effect because an effect of diagnosis on BP_{F} in both small regions (such as raphe nuclei and insula) and large regions (such as parietal and occipital cortices) was detected, and partial volume effects would affect smaller brain regions disproportionately. Furthermore, a partial volume effect would have led to underestimation of BP_{F} in the diagnostic group that was older and/or had the neuropathology found in MDD, but the depressed NRM MDD subjects (which were slightly older and had the MDD pathology) had higher BP_{F} than HV.

The results of the machine-learning analyses supported the use of whole-brain voxel-based 5-HT_{1A} binding maps as biomarkers to distinguish individuals at high risk for developing MDD from healthy individuals. Familial transmission implies that not all offspring of MDD probands should manifest an elevation in 5-HT_{1A} binding and predicts a bimodal distribution of BP_{F} in HR subjects. The multivoxel pattern analysis (MVPA) results were consistent with this hypothesis in that just over half of the HR subjects were classified as HV.

Serotonin-1A (5-HT_{1A}) Receptor Binding Potential (BP_{F}) as Biomarker for Treatment

Also reported herein are result that show that any antidepressant-associated downregulation of 5-HT_{1A} binding reverses within two weeks of medication discontinuation (Examples 6-10).

Since SSRIs are thought to work by reversing the MDD-related elevation in autoreceptor binding (Gray et al., 2013), once this effect wears off, the patient may no longer be protected from a return of symptoms or recurrence of the episode or relapse into a new episode. Consequently, an important question is: how long does the antidepressant effect on the 5-HT_{1A} autoreceptors persist following medication discontinuation? Additionally, this information will tell us how long patients need to be off antidepressant medications before PET scanning can offer a reliable measure of illness-related binding levels. Thus, the relationship of 5-HT_{1A} binding to time since antidepressant medication treatment was discontinued in patients with a DSM-IV diagnosis of MDD was examined, and compared these patients to MDD patients who were medication naïve at time of

These results indicated that any antidepressant-associated downregulation of 5-HT_{1A} autoreceptor binding reversed within two weeks of medication discontinuation. Since this effect is hypothesized to mediate the antidepressant action of SSRIs, and perhaps other antidepressants, it suggests that patients who need ongoing treatment may relapse rapidly when medication is discontinued. Moreover, two weeks appears to be a sufficiently long washout of antidepressant medications for a reliable measure of illness-related binding levels.

No differences were found in [¹¹C]WAY-100635 binding between antidepressant naïve and antidepressant exposed MDD groups. Furthermore, [¹¹C]WAY-100635 binding did not correlate with time off antidepressants in the antidepressant exposed patients over an extended period of time ranging from two to 728 weeks. Moreover, a comparison with a group of MDD patients before SSRI treatment and then after seven weeks of SSRI treatment, and scanned while still on the SSRI (Table 5), showed that pretreatment binding was comparable to binding in both the antidepressant naïve MDD group and in antidepressant exposed MDD when that group has been off antidepressants for at least two weeks. These results suggested that previously described antidepressant-induced downregulation of 5-HT_{1A} receptor binding (Gray et al., 2013) may reverse within as little as two weeks off antidepressant medication.

This finding has implications for clinical care, as this biomarker of antidepressant action indicates a loss of antidepressant effect in that time frame. This is important as patients with MDD often discontinue treatment but are not forthcoming about it. The observation also validates the use of PET to study disease-related receptor binding after two weeks of medication washout. The groups were comparable with regard to free fraction estimation using f_{P} and both groups received comparable dose of radiotracer (Table 5). The results were primary reported as BP_{F} but did not differ from results using alternative outcome measures of BP_{P} and BP_{ND}. This indicated the findings were quite robust in terms of outcome measure, of importance for future studies that rely on these findings for research design in terms of patient medication status.

As shown in Table 7 raphe and hippocampal binding in medication naïve and medication washed out MDD groups were comparable with the pretreatment binding in MDD in Gray et al. The SSRI treatment group shows a 17% decline in binding after an average of 7 weeks of SSRI medication and the binding was also lower compared with medication naïve and medication washed out MDD groups. The results reported herein show that discontinuation of SSRIs result in a return of depressive symptoms in as little as two weeks.

Methods of Detecting Serotonin-1A (5-HT_{1A}) Receptor Binding Potential (BP_{F})

Any method that can detect elevated serotonin 1A (5-HT_{1A}) receptor binding potential (BP_{F}) in the brain of a subject can be utilized in the methods of the invention. These methods include but are not limited to radioligand-labeled positron emission tomography (PET) and single-photon emission computed tomography (SPECT).

In some embodiments, a radioligand capable of binding with a serotonin 1A receptor is introduced into the subject. In some embodiments, the radioligand is introduced by injection into the bloodstream.

In some embodiments, the analysis of the PET or SPECT images is a computer analysis. In some embodiments, the analysis is multivoxel pattern analysis (MVPA).

In some embodiments, method further comprises carrying out one or more magnetic resonance image (MRI) scans of the subject. In some embodiments, the MRI images are analyzed to define the boundaries of the regions of interest (ROIs).

Distribution, density and activity of receptors in the brain can be visualized by radioligands labeled for SPECT and PET, and the receptor binding can be quantified by appropriate tracer kinetic models, which can be modified and simplified for particular application. Selective radioligands are available for the various transmitter systems, by which the distribution of these receptors in the normal brain and changes in receptor binding during various physiologic activities or resulting from pathologic conditions can be visualized.

A radioligand needs to contain a radioisotope that can be selected from the group consisting of ³H, ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁴⁴Sc, ⁶⁷Ga, and ⁶⁸Ga. The preferred radioisotopes are ¹¹C and ¹⁸F.

Radioligands that can be used to detect the serotonin-1A (5-HT_{1A}) receptor binding potential (BP_{F}) include but are not limited to antagonist PET tracers, including but not limited to radiolabeled N-[2-[4-(2-methoxyphenyl)-1-piperazinyl]ethyl]-N-2-pyridinylcyclohexanecarboxamide (WAY100635). WAY100635 can be radiolabeled with carbon-11. In some embodiments, the WAY 100635 can be radiolabeled at the carbonyl carbon and in other embodiments at the methyl carbon of the methoxy group. In some embodiments, the WAY100635 can be labeled with ¹⁸F.

Other antagonist PET tracers that can be used in the method include desmethyl-WAY100635, reverse amide of WAY1060035, [¹⁸F]MPPF, a fluorophenyl analogue of WAY100635, synthesized by the nucleophilic displacement of the corresponding nitro precursor with [¹⁸F]fluoride, and [¹⁸](cis)-FCWAY, a fluoro analogue of WAY100635, and ¹⁸F]MeFWAY, a fluoromethyl analogue of WAY100635.

Agonist PET tracers can also be used in the methods of the invention and include but are not limited to [¹¹C]MPT, an arylpiperazine derivative of 3,5-dioxo-(2H,4H)-1,2,4-triazine, [¹¹C] 2-(4-(4-(2-methoxyphenyl)piperazin-1-yl)butyl)-4-methyl-1,2,4-triazine-3,5 (2H, 4H) dione (CUMI-101), and [¹¹C]MMP.

The serotonin-1A (5-HT_{1A}) receptor binding potential (BP_{F}) is detected in the brain, and can be detected in at least one region including but not limited to: the anterior cingulate; amygdala; cingulate body; dorsolateral prefrontal cortex; hippocampus; insula; medial prefrontal cortex; occipital lobe; orbital prefrontal cortex; parietal lobe; parahippocampal gyrus; temporal lobe; and raphe nuclei.

In some embodiments, the serotonin 1A (5-HT_{1A}) receptor binding potential (BP_{F}) is detected in all regions of interest.

In some embodiments, the reference value of receptor binding potential is the value of receptor binding potential of a radioligand to serotonin 5-HT_{1A} receptors in the brain of a healthy control. In some embodiments, reference value of receptor binding potential is the value of receptor binding potential of a radioligand to serotonin 5-HT_{1A} receptors in the raphe (the region from which all serotonergic neurons originate).

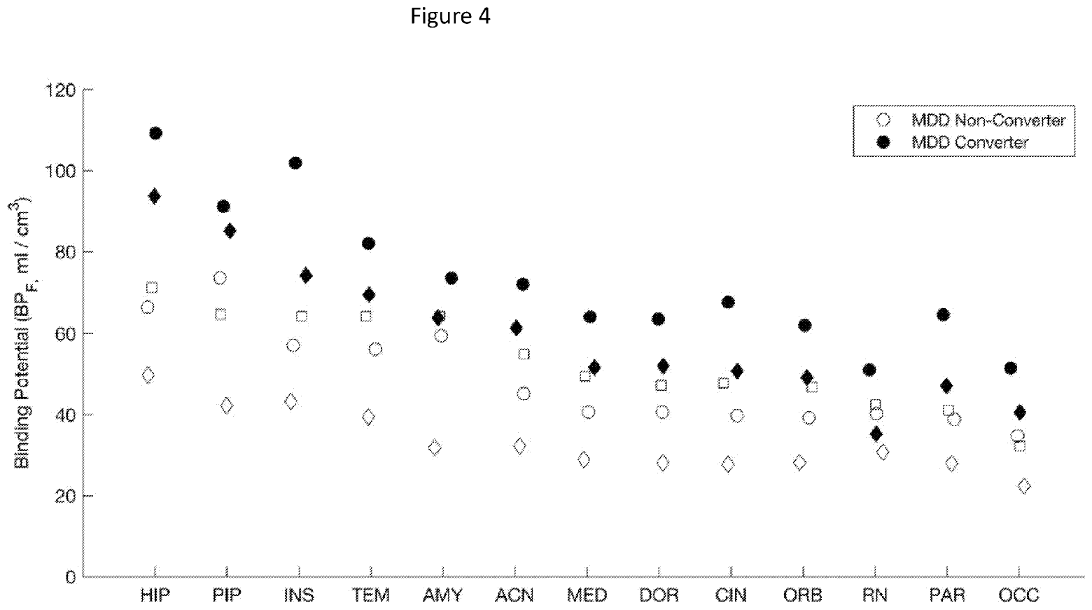

As shown in Example 5 and FIG. 4, offspring of parents of MDD who converted to subjects with MDD had the highest serotonin 1A receptor binding potential across most ROIs of the brain. Thus, in some embodiments, the reference value of the offspring who developed MDD can be used in the methods of the invention. In some embodiments, this reference value is equal or greater than about 80 ml/cm³. In some embodiments, this reference value is equal or greater than about 70 ml/cm³. In some embodiments, this reference value is equal or greater than about 60 ml/cm³. In some embodiments, this reference value is equal or greater than about 50 ml/cm³.

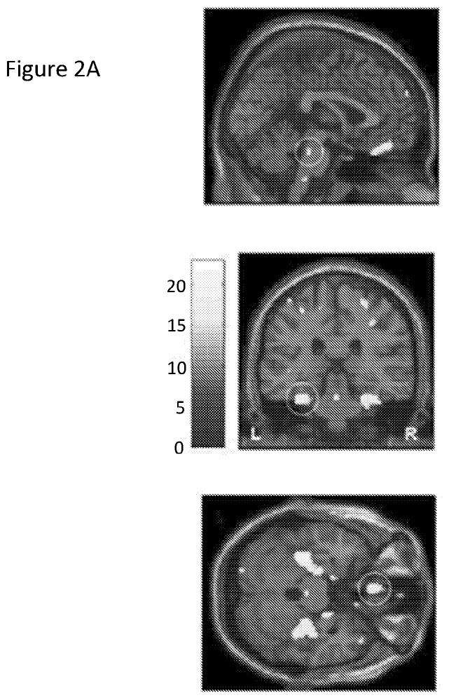

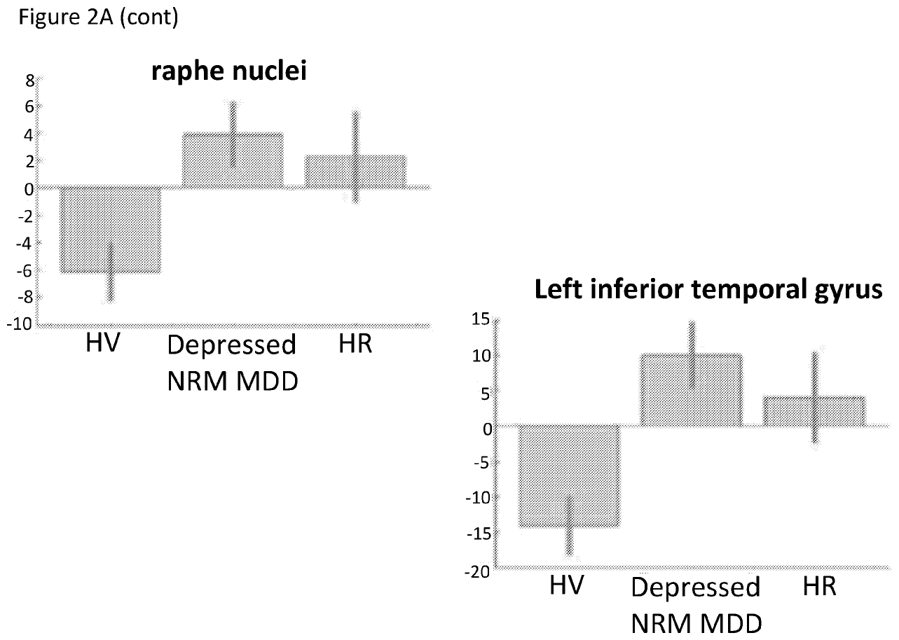

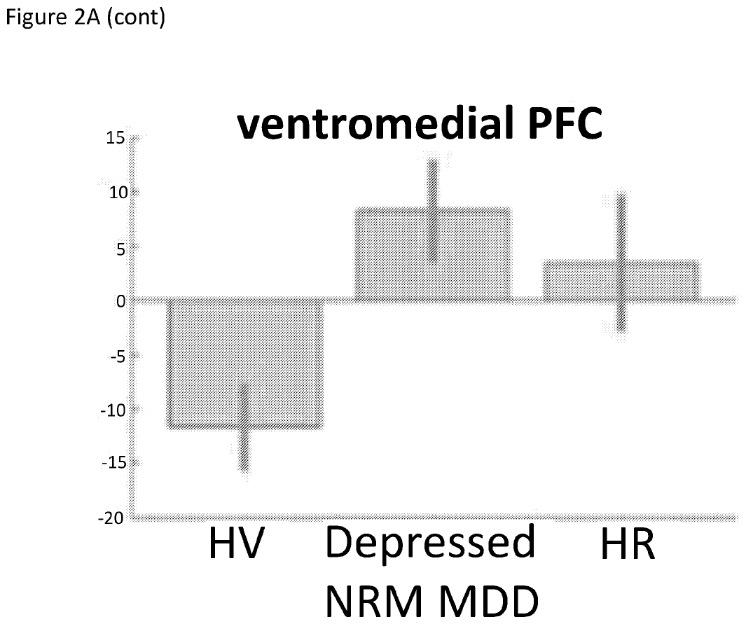

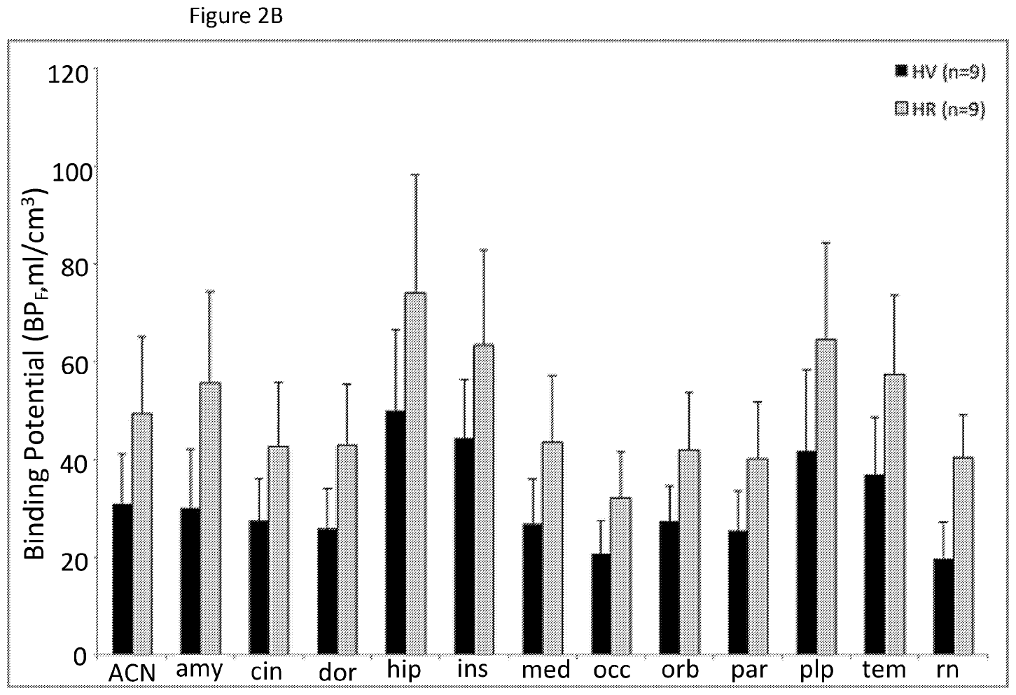

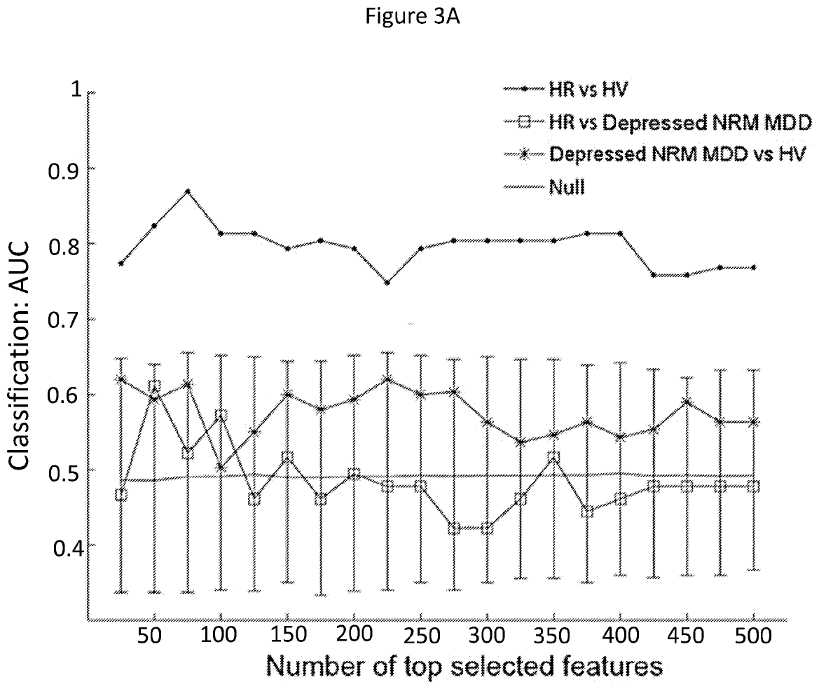

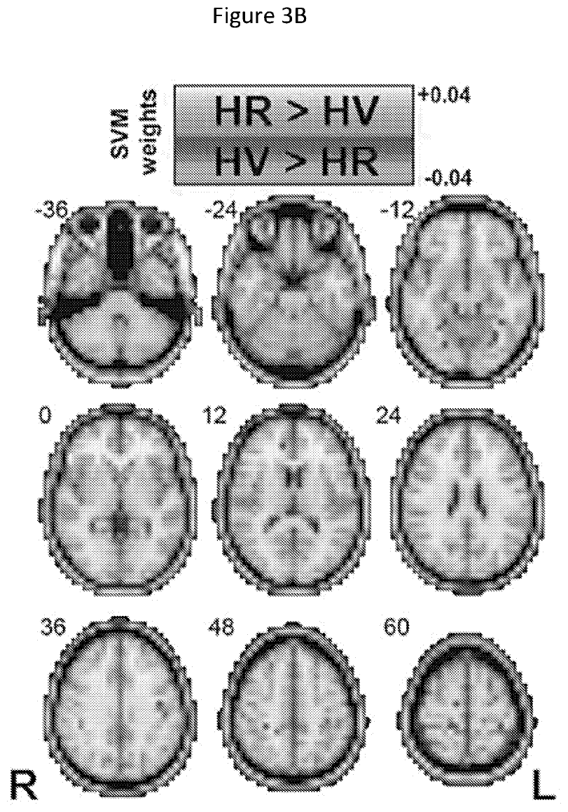

As shown in Example 4 and FIGS. 2 and 3, subjects with serotonin 1A receptor binding potential pattern in the brain the same or similar to a subject with MDD converted to subjects with MDD. Thus, in some embodiments, a comparison of the serotonin 1A receptor binding potential pattern in the brain of the subject to the pattern in subjects with MDD can be used in the methods of the invention.

In some embodiments, the free fraction (f_{P}) of a PET tracer, measured from a single venous sample is used in the calculation of BP_{F} (Milak, et al., 2010a). The f_{P} value for [^{Carbonyl-11}C]WAY-100635 is directly correlated with 5-HT_{1A} BP_{F} in the raphe nucleus (RN) and [^{Carbonyl-11}C]WAY-100635 acts as a surrogate endophenotype for increased binding and a high risk of MDD. With a direct correlation present, simple venous sampling following administration of [^{Carbonyl-11}C]WAY-100635 allows for its use in the methods of the invention.

As shown in Example 1 and Table 1, HV subjects had a percent free fraction of PET tracer of about 8% where Depressed NRM MDD had a percent free fraction of PET tracer of about 6.2%. Thus, in some embodiments the percent free fraction of PET tracer can be used as a reference value and if the subject has a percent free fraction at about or below the reference value, they are identified as higher risk for MDD and should be treated. In some embodiments, the reference value is about 6.2% free fraction of PET tracer. If a subject has a percent free fraction about at or below 6.2% they are identified at higher risk for MDD and are candidates to be treated.

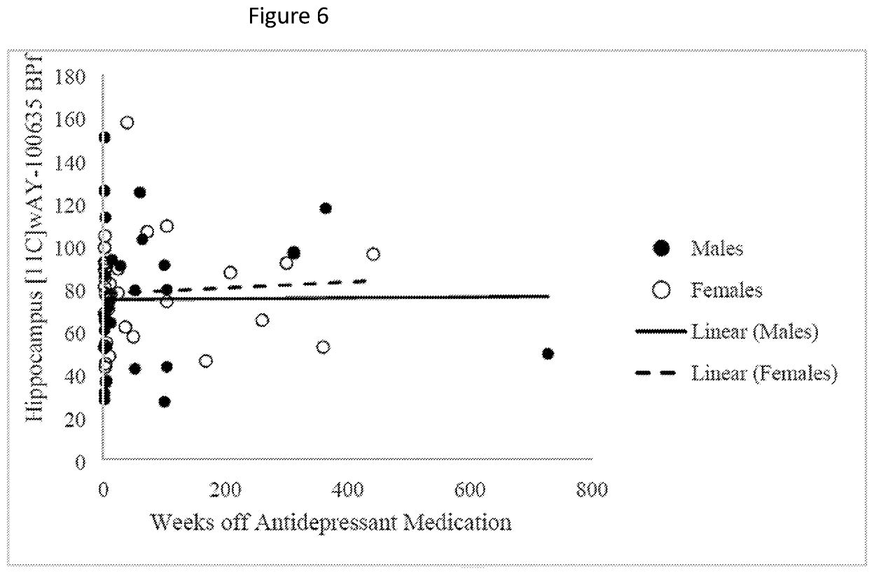

As shown in Example 9 and FIG. 6, subject who had been off treatment for MDD had higher serotonin 1A receptor binding potential across most ROIs of the brain. Thus, in some embodiments, the reference value of subjects who ceased treatment after two weeks can be used in the methods of the invention. In some embodiments, this reference value is equal or greater than about 80 ml/cm³.

As shown in Example 10 and Table 7, subjects who had been off treatment for MDD had higher serotonin 1A receptor binding potential across most ROIs of the brain. Thus, in some embodiments, the reference value of subjects who ceased treatment after two weeks can be used in the methods of the invention. In some embodiments, this reference value is equal or greater than about 40 ml/cm³.

Methods of Treating Subjects with MDD

One major advantage of the present invention is it allows the treatment of individuals with MDD before symptoms have occurred. Once a subject has symptoms of MDD, detrimental changes in their brain have occurred and they may be less likely to respond to treatment and less likely to adhere to a treatment regimen. But, even fully symptomatic persons can respond to treatment and showed a conversion from the MDD brain biological picture of higher binding to lower binding, only so long as they continue treatment. See Examples 6-10. Thus, the method of the invention includes treatment of subjects with the endophenotype even if they are asymptomatic.

Therapeutic agents for use in the methods of the invention include, but are not limited to, selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine or monoamine oxidase inhibitors (MAOIs).

The antidepressants for use in the methods of the invention include but are not limited to Citalopram, Escitalopram, Paroxetine, Fluoxetine, Fluvoxamine, Sertraline, Desvenlafaxine, Duloxetine, Levomilnacipran, Milnacipran, Venlafaxine, Tratnadol, Sibutramine, Etoperidone, Lubazodone, Nefazodone, Trazodone, Atomoxetine, Reboxetine, Viloxazine, Bupropion, Amphetamine, Dextroamphetamine, Dextromethamphetamine, Lisdexamfetamine, Amitriptyline, Butriptyline, Clomipramine, Desipramine, Dosulepin, Doxepin, Imipramine, Iprindole, Lofepramine, Melitracen, Nortriptyline, Opipramol, Protriptyline, Trimipramine, Amoxapine, Maprotiline, Mianserin, Mirtazapine, Isocarboxazid, Phenelzine, Selegiline, Tranylcypromine, Moclobemide, Pirlindole, Mianserin, Mirtazapine, Vilazodone, Vortioxetine, Tandospirone, Quetiapine, and AZD6765.

Selection of a therapeutically effective dose will be determined by the skilled artisan considering several factors, which will be known to one of ordinary skill in the art. Such factors include the therapeutic agent, and its pharmacokinetic parameters such as bioavailability, metabolism, and half-life. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

The methods of the invention can also include psychotherapy. In some embodiments, the psychotherapy is selected from the group consisting of Interpersonal Therapy and Cognitive Behavioral Therapy.

As shown in Examples 6-10, patients treated for MDD show a change in the pattern of high 5-HT_{1A} binding to low 5-HT_{1A} binding again proving that treating these asymptomatic patients for MDD who have the biomarker/endophenotype of high 5-HT_{1A} binding is viable and important to clinical success.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Methods and Materials for Example 2-4

Subjects

Data from nine healthy high risk (HR), 30 unrelated depressed not recently medicated (NRM) subjects with major depressive disorder (MDD) and 18 remitted NRM MDD subjects, and 51 healthy volunteers (HV) were analyzed for this study.

The data from HV and all NRM MDD subjects are presented for comparison purposes but were previously published (Kaufman et al., 2015; Parsey et al., 2010a). The HR subjects were recruited and their PET data were acquired contemporaneously with the comparison groups (i.e., HV, depressed NRM MDD and remitted NRM MDD). Subjects were recruited through printed and online advertisements and referral from our clinical populations.

Subjects were classified as HR if they had no lifetime or current history of a DSM-IV psychiatric illness based on a structured clinical interview (SCID I) (First et al., 1995) and had one or more first-degree relatives with a history of early onset (<30 years of age) MDD. Five subjects reported having one first-degree relative with a history of MDD (a parent in all cases), and four reported having two or more (i.e., parent, sibling). No subject confirmed a history of depression in both parents. All subjects provided consent for the Principal Investigator to contact their relative(s) with a history of MDD to confirm their reports. Research interviews were conducted by clinical raters holding Master's degrees or higher. Assessment instruments used for ascertaining family history of mood disorders included a baseline demographic interview, the Childhood Experiences Questionnaire (CEQ)-Modified Abuse History, the Family History for Genetic Studies (FIGS), and the Parental Bonding Instrument (PBI).

Additional inclusion criteria included the following: being between the ages of 18 to 32 years; absence of history of treatment with psychotropic medication; and taking no other medications impacting the serotonin system for a minimum of 6 months or any anticoagulant medication for a minimum of 10 days. Exclusion criteria consisted of the following: current or past MDE or other Axis I psychiatric diagnosis; current or past alcohol or drug use disorder; history of IV drug use or ecstasy use more than twice; family history of schizophrenia; significant active physical illness; inability to consent; pregnancy; presence of metal implants or a medicinal patch; medical or occupational radiation exposure within the past 12 months; or a head injury causing loss of consciousness for more than three minutes. New York State Psychiatric Institute/Columbia Institutional Review Board-approved written informed consent was obtained from all subjects after they were given a description of the study.

Radiochemistry and Input Function Measurement

Preparation of [¹¹C]WAY100635 was performed as previously described (Parsey et al., 2000). Between 96.2 and 732.6 MBq of [¹¹C]WAY100635 were injected (Table 1). Mean injected mass (μg) differed across groups (F=9.00, df=2, 87, p<0.001); a pairwise post hoc test revealed that the HV group received higher mass than depressed NRM MDD and HR, which received comparable mass. Although it has been shown that injected mass in this range does not correlate with binding potential (Miller et al., 2009), the injected mass was adjusted in the analyses.

Arterial plasma radioactivity, metabolites, and plasma free fraction (f_{P}) were collected and assayed as previously described (Parsey et al., 2006; Parsey et al., 2000). Unmetabolized parent fraction levels were fit with a Hill function (Wu et al., 2007). The input function was corrected for unmetabolized tracer by multiplying the total plasma counts with the interpolated parent fraction. The metabolite-corrected arterial input function was fit as the combination of a straight line and the sum of three decreasing exponentials, describing the function before and after the peak, respectively.

TABLE 1
Tracer Dose and Free Fraction in High Risk (HR), Healthy Volunteer (HV) and Depressed
Not-Recently Medicated (NRM) Major Depressive Disorder (MDD) Subjects

| | | | | p-values | |
|---|---|---|---|---|---|
| | | | | | HR vs. |
| | | | Depressed | | Depressed |
| | HR | HV | NRM MDD | HR vs. HV | NRM MDD |
| N | 9 | 51 | 30 | | |
| Percent | 6.50 ± 1.62% | 8.10 ± 2.39% | 6.15 ± 1.72% | 0.059 | 0.596 |
| Free | | | | | |
| Fraction | | | | | |
| Injected | 213.49 ± 41.44 | 295 ± 126.91 | 230 ± 97.31 | 0.060 | 0.614 |
| Dose (MBq) | | | | | |
| Injected | 1.62 ± 1.26 | 2.98 ± 1.94 | 1.49 ± 0.92 | 0.050 | 0.721 |
| Mass (μg) | | | | | |

Values are mean ± SD

Image Acquisition and Analysis

PET image acquisition protocol details have been previously described (Parsey et al., 2010a; Parsey et al., 2006; Parsey et al., 2000). Briefly, venous and arterial catheters were used to inject radiotracer and to obtain arterial samples for the input function, respectively. The head was immobilized using a polyurethane head holder system (Soule Medical; Tampa, Fla., USA). PET imaging was performed using an ECAT Exact HR+(Siemens/CTI; Knoxville, Tenn., USA). Data were collected in 3D mode for 110 minutes in 20 frames of increasing duration: 3 at 20 seconds, 3 at 1 minute, 3 at 2 minutes, 2 at 5 minutes and 9 at 10 minutes.

Images were reconstructed, using attenuation correction from the transmission data, to a 128×128 matrix (pixel size: 1.72×1.72 mm). A model-based method was used to correct scatter (C C et al., 1996). A Shepp filter of 0.5 (2.5 mm in full width at half maximum, FWHM) was used for the reconstruction and estimated image. The Z filter was all-pass 0.4 (2.0 mm in FWHM), and the zoom factor was 4.0, leading to a final image resolution of 5.1 mm at FWHM at the center of the field of view (Mawlawi et al., 2001).

The last 12 frames of each study were registered to the eighth frame using the FMRIB linear image registration tool (FLIRT) version 5.0 (FMRIB Image Analysis Group, Oxford, UK). Linear co-registration was performed between the averaged motion-corrected PET frames and the MRI as previously described (DeLorenzo et al., 2009).

Acquisition of T 1-weighted MRI images for co-registration of PET images and identification of regions of interest (ROIs) were performed as previously described using a 3T Signa HDx system (General Electric Medical Systems; Milwaukee, Wis., USA) (Milak et al., 2010). Regional delineations were obtained automatically for all ROIs except for dorsal raphe nucleus (RN), which was manually located on each PET image and delineated by a fixed-volume (20 mm3) elliptical ROI (Parsey et al., 2010a; Parsey et al., 2006). Automatic ROIs were obtained using nonlinear registration techniques to warp 18 manually outlined MRIs. The 18 templates were registered to the skull-stripped (using Atropos, (Avants et al., 2011)) target brain MRI using the Automatic Registration Toolbox (ART (Ardekani et al., 2005)), which was a top performer in an evaluation of 14 nonlinear brain registration algorithms (Klein et al., 2009). The probabilistic regional label for each target voxel was then determined by evaluating the percentage of the 18 normalized brains assigning that regional label to the voxel. For cortical regions, this probability was multiplied by the probability of the voxel being in the grey matter, as determined by SPMS (Wellcome Trust Centre for Neuroimaging, London, UK). The labels are therefore probabilistic and these probabilities were used in the calculation of the time-activity curves (TACs). MRI-based ROIs were applied to each frame of the PET images using the co-registration transformation. TACs were then generated by averaging the measured activity within a region over the time course of the PET acquisition.

The PET outcome measure of interest most closely reflecting Bₐᵥₐᵢₗ and requiring the fewest assumptions is BP_{F} (=Bₐᵥₐᵢₗ/K_{d}) (Innis et al., 2007). A full description of the modeling approach used to quantify BP_{F} has been previously described (Parsey et al., 2010a; Parsey et al., 2001). Briefly, at the ROI level, the TACs were fitted using a constrained kinetic two-tissue compartment model (i.e., constraining the non-displaceable binding in each target region to be equal to the tracer distribution volume in the RR (Parsey et al., 2000)). The cerebellar white matter (CWM) RR was selected because it has the least specific binding of all available options and was fitted with a one-tissue compartment model (Parsey et al., 2005). BP_{F} was calculated as (V_{T(ROI)}-V_{T(RR)})/f_{P}, where VT is the total volume of distribution in the specified region.

At the voxel level, BP_{F} was estimated using the basis pursuit strategy (Gunn et al., 2002), which provides parametric images from dynamic radiotracer data without the need to specify a compartmental structure. Briefly, this data-driven approach is based on the general compartmental theory for description of the radiotracer's kinetics and determines a parsimonious model consistent with the measured data. This approach uses basis pursuit denoising, a technique that involves the determination of a sparse selection of kinetic basis functions from an overcomplete dictionary to compromise between the error in the description of the measured data and the sparseness of the representation. This approach provides estimates of the system's macro parameters (i.e., V_{T}) and the corresponding number of numerically identifiable compartments in the system. This approach determines the most appropriate model from the information contained within the measured data and requires no a priori knowledge of the radiotracer kinetics, besides the choice of the family of basis functions that constitutes the dictionary, which needs to be in a range physiologically plausible for the considered radiotracer. Here, a range spaced in a logarithmic manner was used to elicit a suitable coverage of the kinetic spectrum, as suggested for the radiotracer [¹¹C]WAY100635 (Gunn et al., 2002). Once the VT parametric images were obtained in each subject using the basis pursuit strategy, the corresponding BP_{F} images were generated by subtracting in each voxel the VT of the CWM-validated as RR for [¹¹C]WAY100635 (Parsey et al., 2000)—and dividing by f_{P}. All methods were implemented by our team in MATLAB (R2009b, The MathWorks, Natick, Mass.).

ROI-Based Statistical Analyses

Statistical analyses were performed in R 3.1.2. Linear mixed effects models incorporated outcome measure data from all ROIs simultaneously, with ROI as the fixed effect and subject and date of experiment as the random effects. By considering all ROIs simultaneously, statistical power was gained and the issues of multiple comparisons avoided. Covariates were included as fixed effects as needed.

The validity of classical inferential statistics such as analysis of variance (e.g., with a mixed effects model) is contingent upon the data tested fulfilling all the assumptions of the test applied (i.e., normality of distribution, compound symmetry, sphericity, kurtosis) (Carlson et al., 2013; Dunn and Clark, 1987; Pantazatos et al., 2012). Power transformations are a standard set of mathematical data manipulations that are applied based on the findings of the residual analysis to transform the data so that it fulfills the assumptions mentioned above. Log transformation of the imaging data yielded normal distribution meeting the requirements for the analysis of variance.

Additionally, bootstrap errors were calculated for each subject for every ROI observation that took into account the error in modeling the metabolite, plasma, and TACs (Ogden and Tarpey, 2006). All observations were weighted according to the calculated bootstrap error. Partial volume correction was not performed because such correction adds noise to the data.

Additional statistical analyses performed include Student's t-test, Fisher's exact test, and chi-squared tests performed in SPSS 19.0 (IBM, SPSS Statistics, 2011).

Voxel-Based Univariate Analyses and Multivariate Machine Learning-Based Analyses

Voxel-level analyses were conducted to validate ROI findings and to explore brain-wide for other regions of difference. Voxel-level PET (BP_{F}) maps were spatially normalized using ART and interpolated to 2×2×2 mm voxel resolution, smoothed with an 8 mm Gaussian kernel, and submitted to a 1-way ANCOVA with three levels: HV, N=51; depressed NRM MDD, N=29; HR, N=9 (due to excessive cropping and difficulties in spatial normalization, 1 depressed NRM MDD subject who was included in the above ROI analyses was dropped from subsequent voxel-based analyses). An absolute threshold was applied to remove voxels with BP_{F} values below 5, and non-grey matter voxels were excluded from analyses via a grey matter mask generated by thresholding a tissue probability map in MNI space (provided with SPM8) at >0.2. Covariates were entered stepwise and non-significant covariates were removed. Sex was included as a nuisance covariate, and these effects were removed from features (voxel BP_{F} values) prior to classification analyses using multivoxel pattern analysis (MVPA, see below). Analyses were conducted using SPM8 (www.fil.ion.ucl.ac.uk/spm/) and implemented in MATLAB version 7.13 on Ubuntu Linux OS 12.0.4.

MVPA analyzes the joint BP_{F} signal across multiple regions in a single subject in order to predict the diagnostic class of that subject. There is no direction associated with the predictions (i.e., class labels are arbitrarily positive or negative). To make MVPA computationally tractable and reduce dimensionality, PET maps were resampled from 2×2×2 mm voxel resolution to 6×6×6 mm resolution. For all binary classification tasks, a linear kernel Support Vector Machine SVM (Vapnik, 1999) with a filter feature selection (t-test) and leave-one-out cross-validation was applied using the Spider v1.71 MATLAB toolbox (http://people.kyb.tuebingen.mpg.de/spider/) with default regularization parameter C=1. During each iteration of leave-one-out cross-validation, one subject was withheld from the data set and: (a) a 2-sample t-test was performed over the remaining training data; (b) the features were ranked by absolute t-score and the top 25, 50, 75 . . . to 500 features were selected; (c) these selected features were then used to predict the class of the withheld test examples during the classification stage. Classification performance is reported in terms of “area under the curve” (AUC), i.e., area under the receiver operator characteristic (ROC) curve (Hanley and McNeil, 1982), which is the arithmetic mean of sensitivity (true positive rate) and specificity (true negative rate). Significance was assessed using permutation as described in Golland and Fischl (2003). P-values for the peak AUC values were calculated with respect to the 4,000 null values obtained above, and corrected p-values were obtained by Bonferroni correction for the number of comparisons (in this case 20, corresponding to the top 25, 50, 75 . . . 500 selected features). It is noted that this approach (plotting performance vs. number of selected features) was not biased, since for each top selected number of features, and for each round of leave-one-out cross validation, the features were selected from a training set (i.e., total number of samples minus one) that was completely independent from the testing set (i.e., one left out sample). Analyses were also conducted whereby models trained to discriminate depressed NRM MDD vs. HV and remitted NRM MDD vs. HV were applied to HR subjects and compared to follow-up data (i.e., were MDD converters classified as MDD or HV). For display purposes, the top 75 features in classifying HR vs. HV were selected from and used to train a model over the entire dataset in order to estimate and display their SVM weights on the brain.

### Example 2—Demographic and Clinical Results for the Subjects

HR subjects do not differ from HV in sex ratio (p=0.94) or lifetime aggression severity (p=0.68) but were younger because younger subjects who had not passed the age of risk were deliberately recruited (p=0.02; Table 2). Similarly, HR show no differences from the depressed MDD group in sex ratio (p=0.27) and lifetime aggression severity (p=0.10) but were younger than the depressed NRM MDD group (p=0.002). The Hamilton Depression Rating Scale (HDRS) scores of the HR subjects were lower than those of the depressed NRM MDD group (p<0.001) and slightly higher than those of HV (p=0.006) but still within normal range.

TABLE 2
Demographic and Clinical Features in High Risk (HR), Healthy Volunteer (HV) and
Depressed Not-Recently Medicated (NRM) Major Depressive Disorder (MDD) Subjects

| | | | | | | | p-values | |
|---|---|---|---|---|---|---|---|---|
| | | | | | | | | HR vs. |
| | | | | | Depressed | | | Depressed |
| | HR | | HV | | NRM MDD | | HR vs. HV | NRM MDD |
| N | 9 | | 51 | | 30 | | | |
| Age (mean ± SD) | 25.5 ± 3.4 | | 37.4 ± 14.5 | | 40.6 ± 13.1 | | 0.02 | 0.002 |
| Female (N \| %) | 5 | 55.6% | 29 | 56.9% | 22 | 73.3% | 0.94* | 0.27* |

| | | | | | |
|---|---|---|---|---|---|
| Aggression (mean ± SD) | 15.4 ± 3.3 | 14.90 ± 3.7 | 18.5 ± 5.1 | 0.68 | 0.1 |
| HDRS (mean ± SD) | 4 ± 2.7 | 0.7 ± 1.0 | 26.1 ± 0.4 | 0.006 | 3.7E−16 |

*denotes Fisher's Exact Test.

### Example 3—ROI-Based 5-HT_{1A} Binding Potential (BP_{F})

The methods described in Example 1 were used.

A main effect of diagnosis was found for BP_{F} tested simultaneously across all 13 ROIs between HV, HR, and depressed NRM MDD subjects (F=11.54, df=2, 87, p<0.0001; FIG. 1A and FIG. 1B). This finding was preserved when corrected for sex, age, injected mass and dose (F=8.1, df=2, 83, p=0.0006).

Post hoc testing showed that the HR group has higher BP_{F} compared with HV (F=8.69, df=1, 87, p=0.004; FIGS. 1A and 1B) but did not differ from the depressed NRM MDD group (F=0.021, df=1, 87, p=0.884). Average BP_{F} in HR subjects was 49.9%±11.8% higher than that in HV and comparable to that in depressed NRM MDD subjects. There was no difference in binding between HR subjects with one first degree relative with a history of MDD versus those with two or more such relatives.

To confirm that the reference region difference did not account for the findings, an ANOVA was performed to evaluate the main effect of diagnosis on VT(RR) and found VT(RR) did not differ across groups (F=0.093, df=2, 86, p=0.911).

### Example 4—Voxel-Based 5-HT_{1A} Binding Potential (BP_{F})

The methods described in Example 1 were used.

Voxel-based analysis showed an effect of diagnosis on 5-HT_{1A} BP_{F} between HV, HR, and depressed NRM MDD subjects (correlation between ROI- and voxel-based results: R²=0.911; slope=0.865, FIG. 2A, left side, Table 3). BP_{F} in HR and depressed NRM MDD subjects vs. HV was highest in midbrain, parahippocampal gyms, and ventral prefrontal cortex (FIG. 2A, right side).

MVPA showed that voxel-based 5-HT_{1A} BP_{F} maps contained sufficient information to distinguish HR versus HV with well above chance performance (even after correcting for sex; mean AUC=0.80, p<0.005, FIG. 3A, Table 4). The highest discrimination was obtained between HR vs. HV using the top 75 selected features (peak AUC=0.87,p<0.0005, sensitivity=0.78, specificity=0.96). The depressed NRM MDD subjects were modestly distinguishable from HV at 25 features (peak AUC=0.62, p=0.08) but not at >25 features, and the mean AUC (across all top N selected features) was not significant (p=0.17). The HR were indistinguishable from depressed NRM MDD subjects across all features (mean AUC=0.49, p=0.43). High HR versus HV classification accuracy remained after correcting for age and sex (i.e., after removing the variance explained by age and sex) (mean AUC=0.72, p<0.005, peak AUC=0.81, p=0.00025, data not shown), as well as after correcting for age, sex and injected mass (mean AUC=0.62, p=0.03, peak AUC=0.80, p=0.00025). Informative voxels discriminating HR vs. HV are displayed in FIG. 3B. Repeating the MVPA using a balanced sub-sample of HV and depressed NRM MDD subjects matched for age and sex (N=9 each group) showed that HR remained distinguishable from HV (mean AUC=0.69, p=0.03; FIG. 2B) and indistinguishable from depressed NRM MDD subjects (mean AUC=0.55, p=0.27), and HV were distinguishable from depressed NRM MDD subjects (mean AUC=0.77, p=0.02) (data not shown).

Owing to the relatively low classification rates of HV vs. depressed NRM MDD groups (when using the full depressed NRM MDD dataset), an additional MVPA analysis was conducted of HV versus an independent group of remitted NRM MDD subjects (N=18, mean (SD) age=34.8 (12.7), 13 females) under the assumption that the remitted NRM MDD subjects would be more similar to non-depressed HR subjects and also have less globally correlated BP_{F}. MVPA performance was found to be higher when classifying remitted NRM MDD vs. HV controlling for sex (peak balanced accuracy=0.78, sensitivity=0.67, specificity=0.9, p<0.05 corrected, data not shown).

TABLE 3
Univariate SPM Analysis Results (omnibus F-contrast
using family-wise error rate (FWE) correction
at p < 0.05, cluster size (k) ≥ 10)

| Region | x | y | z | size | F-value |
|---|---|---|---|---|---|
| Medulla | 2 | −32 | −50 | 12 | 16.5 |
| Middle Temporal Gyrus | 32 | 2 | −48 | 174 | 17.5 |
| Temporal_Inf_L | −42 | 6 | −36 | 220 | 17.8 |
| Fusiform_R | 36 | −64 | −16 | 1458 | 22.8 |
| ParaHippocampal_R | 20 | −6 | −32 | 147 | 19.8 |
| Fusiform_L | −32 | −34 | −26 | 352 | 22.8 |
| Orbital Gyrus | −6 | 44 | −30 | 208 | 16.6 |
| Amygdala_L | −14 | −4 | −28 | 20 | 15.3 |
| Orbitofrontal_Cortex_R | 8 | 18 | −28 | 13 | 15.0 |
| Temporal_Pole_Sup_R | 46 | 22 | −20 | 49 | 16.2 |
| Pons | 0 | −28 | −24 | 15 | 15.7 |
| Fusiform_L | −32 | −56 | −14 | 388 | 19.5 |
| Temporal_Inf_R | 58 | −48 | −22 | 16 | 15.4 |
| Temporal_Mid_R | 62 | −16 | −18 | 13 | 14.3 |
| Occipital_Mid_R | 46 | −84 | 12 | 120 | 15.0 |
| Sub-Gyral | −38 | −2 | −18 | 245 | 17.7 |
| Cerebelum_6_L | −8 | −70 | −14 | 31 | 15.9 |
| Occipital_Inf_L | −44 | −58 | −14 | 10 | 14.5 |
| Insula | 38 | −12 | −8 | 46 | 16.3 |
| Temporal_Sup_R | 56 | −10 | −2 | 86 | 16.2 |
| Temporal_Mid_R | 54 | −38 | −2 | 44 | 15.6 |
| Temporal_Mid_L | −52 | −40 | −4 | 15 | 14.8 |
| Thalamus_R | 6 | −8 | −2 | 42 | 19.8 |
| Sub-Gyral | −44 | −28 | 0 | 10 | 14.6 |
| Temporal_Sup_L | −48 | −14 | 4 | 38 | 14.6 |
| Frontal_Sup_Medial_L | −4 | 70 | 4 | 11 | 14.9 |
| Frontal_Mid_R | 36 | 62 | 8 | 15 | 14.8 |
| Frontal_Sup_Medial_R | 12 | 66 | 28 | 195 | 17.0 |
| Cingulum_Ant_L | −10 | 32 | 26 | 62 | 16.4 |
| Postcentral_R | 46 | −18 | 36 | 84 | 16.5 |
| Frontal_Sup_Medial_L | −8 | 56 | 42 | 81 | 18.4 |
| Parietal_Inf_L | −36 | −46 | 40 | 125 | 18.2 |
| Postcentral_R | 32 | −36 | 40 | 331 | 22.2 |
| Postcentral_L | −38 | −16 | 42 | 29 | 16.6 |
| Angular_R | 46 | −66 | 44 | 11 | 14.9 |
| Cingulum_Mid_R | 12 | −36 | 44 | 71 | 16.6 |
| Precentral_L | −44 | 6 | 48 | 19 | 15.0 |
| Frontal_Mid_R | 26 | 30 | 46 | 10 | 14.3 |
| Paracentral Lobule | −16 | −36 | 50 | 143 | 17.3 |
| Frontal_Sup_L | −16 | 40 | 48 | 10 | 14.5 |
| Postcentral_L | −32 | −34 | 56 | 60 | 16.4 |
| Precuneus_R | 6 | −44 | 58 | 35 | 15.2 |
| Frontal_Sup_R | 20 | −6 | 56 | 11 | 14.7 |
| Postcentral_R | 24 | −32 | 64 | 50 | 16.7 |
| Postcentral_L | −44 | −30 | 66 | 10 | 15.5 |

TABLE 4
Multivoxel Pattern Analysis (MVPA) Classification
Results in High Risk (HR), Healthy Volunteer (HV) and
Depressed Not-Recently Medicated (NRM) Major Depressive
Disorder (MDD) Subjects (AUC = area under the
receiver operating characteristic curve)

| | mean | mean AUC | | | | |
|---|---|---|---|---|---|---|
| Classification | AUC | p-value | peak AUC | sensitivity | specificity | p-value |
| Depressed | 0.58 | 0.17 | 0.62 | 0.5 | 0.74 | 0.08 |
| NRM MDD | | | | | | |
| (N = 29) vs. | | | | | | |
| HV (N = 51) | | | | | | |
| HR (N = 9) vs. | 0.80 | <0.005 | 0.87 | 0.78 | 0.96 | 0.00025* |
| HV (N = 51) | | | | | | |
| HR (N = 9) vs. | 0.49 | 0.43 | 0.61 | 0.56 | 0.67 | 0.098 |
| Depressed | | | | | | |
| NRM MDD | | | | | | |
| (N = 29) | | | | | | |

*denotes p < 0.05 after Bonferroni Correction.

### Example 5—Diagnostic Follow-Up of Patients in Examples 1-4

Methods

Follow-up was completed during two weeks five to seven years after the PET scan acquisition on five out of nine HR subjects who were reachable for a semi-structured interview (Zimmerman, 1994). Occurrence of a MDE since PET scan acquisition was determined by patient-reported history and treatment and confirmed by the semi-structured interview.

Results

Two of the five HR subjects reported a MDE since the PET scan to date. These subjects had the highest raphe midbrain binding of the five subjects for whom there was follow-up clinical data (FIG. 4). The converters and non-converters cleanly separated on binding in most ROIs and the overall group had an indication of a bimodal distribution consistent with a subgroup having the biologic risk trait.

The predictions of a HV vs. depressed NRM MDD discriminative model applied to the HR subjects were compared with the follow-up data in 5 of the 9 HR subjects. It was hypothesized that the model would classify MDD converters as MDD and the non-converters as HV. It was found that both MDD converters were classified as MDD, whereas one of three non-converters was classified as HV (the same was found when applying a model trained to discriminate HV vs. remitted NRM MDD subjects).

### Example 6—Materials and Methods for Examples 7-10

Participants

Ninety-eight adults had [¹¹C]WAY-100635 PET scans to measure 5-HT_{1A} binding. A subset of these PET data were previously reported on as part of studies of acute or remitted depression (Gray et al., 2013; Miller et al., 2013; Parsey et al., 2006a; Parsey et al., 2010). At the time of study enrollment, all patients (59 females, 39 males, aged 18-70) met DSM-IV criteria for MDD in a current major depressive episode and scored >16 on the 17-item Hamilton Depression Rating Scale (HDRS; Hamilton, 1960). Exclusion criteria included presence of significant active medical conditions, alcohol use disorders or other substance abuse in remission for less than six months, dementia, neurological disease, head injury involving loss of consciousness, pregnancy, first-degree family history of schizophrenia if subject was <33 years old, fluoxetine use within six weeks of PET scanning, or exposure to a 5-HT_{1A} receptor agonist within six months of scanning.

All participants provided informed written consent after an explanation of the study protocol and associated risks as required by the Institutional Review Board.

Measures and Procedures

Subjects diagnosed using the Structured Clinical Interview for DSM-IV (SCID-I) (First, et al., 1995). Interviews were conducted by a doctoral- or masters'-level psychologist. A consensus conference of research psychologists and psychiatrists reviewed and confirmed the diagnosis. Current depression severity was determined using the 17-item Hamilton Depression Rating Scale (HDRS; Hamilton, 1960) and Beck Depression Inventory (BDI; Beck et al., 1961). Suicide history was assessed with the Columbia Suicide History Form (Oquendo et al., 2003).

Psychiatric and medical history, chart review, physical examination, routine blood tests, pregnancy test, urine toxicology, and electrocardiogram were used to ensure eligibility for each study. Study protocols were approved by the Institutional Review Board of the New York State Psychiatric Institute.

Medication history was assessed within two weeks of PET scanning. The number of weeks since last antidepressant exposure was recorded by interview and chart review. This time estimate was less reliable for patients who had been off antidepressants for more than one year. As can be seen from the figure, binding levels were very comparable across the span of 728 weeks. In other words, it will not affect the results if the estimate of the time off of antidepressant medication is less reliable beyond one year. Thirty-two participants were antidepressant naïve at the time of the PET scan, and the remaining 66 subjects had prior antidepressant exposure. Fifty-seven of the 66 medication exposed patients had prior exposure to SSRIs and/or SNRIs. If the participants were on such psychotropic medication at the time of enrollment, they were tapered off the medication and were medication-free for at least two weeks prior to scanning

Radiochemistry and Input Function Measurement

[¹¹C]WAY-100635 was used for quantification of 5-HT_{1A} binding. Details of radiotracer preparation have been described previously (Parsey et al., 2000). A metabolite-corrected arterial input function was obtained (Parsey et al., 2000), and the plasma free fraction (f_{P}) was assayed in triplicate (Parsey et al., 2006b).

Image Acquisition and Analysis

A T1-weighted magnetic resonance image (MRI) scan was acquired for each subject for PET image registration and anatomical labeling, using either a 1.5-T Signa Advantage scanner (General Electric Medical Systems, Milwaukee, Wis.) at a resolution of 1.5×0.9×1.0 mm, or a 3-T SignaHDx scanner at a resolution of 1.0×1.0×1.0 mm.

PET images were acquired with an ECAT EXACT HR+scanner (Siemens/CTI, Knoxville, Tenn.) as previously described (Parsey et al., 2000). After a 15-minute transmission scan, an injection of [11C]WAY-100635 was administered over 30 seconds and then an emission scan of 110 minutes was performed, consisting of 20 frames of increasing duration (3×20 s, 3×1 min, 3×2 min, 2×5 min, 9×10 min).

To correct for between-frame subject motion, each PET frame was registered to the eighth frame of the scan using the FMRIB linear image registration tool (FLIRT), version 5.0 (FMRIB Image Analysis Group, Oxford, UK). Thirteen brain regions of interest (ROIs) were chosen a priori based on areas of abundant binding to the 5-HT_{1A} receptor and included autoreceptors in the raphe nuclei, and terminal field, target neuron receptors in the amygdala, anterior cingulate, posterior cingulate, dorsal prefrontal cortex, hippocampus, insula, medial prefrontal cortex, parietal cortex, parahippocampal gyms, occipital cortex, orbital cortex and temporal cortex as well as the reference region of cerebellar white matter (CWM). All ROIs except for raphe nuclei (RN) were identified on each individual's T1-weighted MRI using a previously described automated probabilistic algorithm (Milak et al., 2010). Because of small size, the raphe nuclei were labeled using a standard space mask of the average location of the raphe nuclei in 52 healthy participants, which was created using [¹¹C]WAY-100635 voxel binding maps as previously described (DeLorenzo et al., 2013). In brief, MRI T1 images were transformed into standardized 3D space using Advanced Normalization Tools (Avants et al., 2014), and the reverse transformation was applied to the raphe nuclei mask.

PET images were co-registered to MRI images using FLIRT, optimized as previously described (DeLorenzo et al., 2009). Time activity curves were generated by plotting the measured activity within a region over the time course of the PET acquisition.

Outcome Measure Estimation

Distribution volumes (VT) of [¹¹C]WAY-100635 were estimated for each ROI using kinetic analysis with an arterial input function and a two tissue compartment constrained (2TCC) model, as has been described elsewhere (Parsey et al., 2000). In brief, time activity curves were fit with a 2TCC model in which the ratio K1/k2 was constrained to that of the reference region (CWM) for each ROI, and BP_{F} was calculated as (V_{T(ROI)}-V_{T(REF)})/f_{P}, where V_{T(ROI)}=distribution volume in the region of interest, VT(REF)=distribution volume in the reference region (CWM), and f_{P} is the radiotracer plasma free-fraction. For comparison purposes, analyses was also run using two additional outcome measures, BP_{P} and BP_{ND}. Note that Table 5 shows that injected dose, mass and specific activity were comparable in the two groups and that free fraction, f_{P} did not differ between groups.

Statistics

Two main analyses were performed. First, a linear mixed effects model was used to compare participants without prior antidepressant medication exposure (n=32) to patients with prior antidepressant medication exposure (n=66) by defining a binary variable indicating past medication status. The same analysis was repeated specifying to patients who had a history of SSRI/SNRI exposure (n=57) and comparing to the medication naïve patients. In order to correct a slight skew in the data, stabilize the variance across regions, and allow for estimates of proportional effects that persist across regions, the analysis was performed on log-transformed estimates of 5-HT_{1A} binding. Log transformation has been used in multiple PET studies by our group and others to address these issues (Hirvonen et al., 2008; Miller et al., 2009; Miller et al., 2013; Parsey et al., 2006a; Parsey et al., 2006b; Parsey et al., 2010; Sullivan et al., 2005).

Observations were weighted according to standard errors calculated based on variation in PET data, plasma data, and metabolite data (Ogden and Tarpey, 2006).

Second, for participants with prior antidepressant medication exposure (n=66), the association between the number of weeks off antidepressants and 5-HT_{1A} binding measured in our a priori regions of interest, was examined using a linear mixed effects model, with weeks off antidepressants as a continuous predictor. Because [¹¹C]WAY-100635 binding has been shown to be dependent on sex (Parsey et al., 2002) and age (Parsey et al., 2002; Tauscher et al., 2001), these covariates were also included in the model as fixed effects. The same analysis was repeated specifying to SSRI/SNRIs exposure in the patients with available data (n=28). These analyses were run with BP_{F} as the main outcome measure of interest and also repeated with BP_{P} and BP_{ND}.

### Example 7—Participant Characteristics

Participants' demographic and clinical data are presented in Table 5. Medication-exposed and medication naïve participants did not differ in age, sex, suicide history, substance use disorder history, comorbid dysthymia or anxiety disorders, depression severity or number of prior episodes of major depression. The antidepressants participants were exposed prior to PET scanning to antidepressants lifetime, include the following classes: selective serotonin reuptake inhibitor (SSRI), serotonin-norepinephrine reuptake inhibitor (SNRI), monoamine oxidase inhibitor (MAOI), tricyclic antidepressant (TCA), atypical antipsychotics, norepinephrine reuptake inhibitor (NERI), mixed reuptake inhibitors (SNRI), and a melatonergic agonist. The classes of antidepressant medications were unavailable for two participants. The distribution of antidepressant medication classes can be found in Table 6. Medication-exposed participants had 1.6 years of education more compared with medication naïve participants (p=0.02). At time of scanning, medication-exposed participants had been off antidepressants for between two and 728 weeks.

Radiotracer dose, mass, specific activity and free fraction did not differ between the two groups (Table 5).

TABLE 5
Clinical Variables, Demographic Variables And Radiotracer Dose

| Clinical Variables, | | | |
|---|---|---|---|
| Demographic | Medication | Medication | p-value (Naïve vs. |
| Variables And | Naïve | Exposed | Exposed, 2-tailed |
| Radiotracer Dose | (n = 32) | (n = 66) | t-test) |
| Weeks Off | N/A | 72.1 ± 133.5 | N/A |
| Antidepressant | | | |
| Medications | | | |
| Age (yrs) | 36.4 ± 13 | 39 ± 12.6 | 0.34 |
| Hamilton Depression | 20.3 ± 5.2 | 19.1 ± 4.5 | 0.23 |
| Rating Scale (17-item) | | | |
| Beck Depression | 25.9 ± 10.1 | 26.4 ± 10.5 | 0.83 |
| Inventory | | | |
| Age of Onset | 22.6 ± 12.5 | 21.6 ± 11.9 | 0.73 |
| Number of Previous | 5.6 ± 9.9 | 6.4 ± 9 | 0.70 |
| Depressive Episodes | | | |
| Length of Current | 176.3 ± 388.5 | 165.7 ± 352.6 | 0.91 |
| Major Depressive | | | |
| Episode (wks) | | | |
| Injected Dose, mCi | 6.33 ± 2.51 | 6.77 ± 3.16 | 0.49 |
| Injected Mass, μg | 1.36 ± 1.00 | 1.67 ± 1.09 | 0.18 |
| Specific Activity, | 2.56 ± 1.22 | 2.10 ± 0.98 | 0.05 |
| mCi/nmol | | | |
| Plasma free fraction | 0.06 ± 0.02 | 0.07 ± 0.02 | 0.10 |
| (fp) | | | |

| | | | | | p- value (Naïve vs. |
|---|---|---|---|---|---|
| | | | | | Exposed, Fisher's |
| Categorical Variables | N (%) | | N (%) | | Exact) |
| Males | 11 | (34) | 28 | (42) | 0.51 |
| Suicide Attempters | 9 | (28) | 21 | (32) | 0.82 |
| Co-morbid Anxiety | 12 | (38) | 27 | (41) | 0.83 |
| Disorder | | | | | |
| Co-morbid Dysthymia | 1 | (3) | 3 | (5) | 1.00 |
| Remitted Alcohol or | 3 | (9) | 10 | (15) | 0.77 |
| Cannabis Dependence | | | | | |
| Race/Ethnicity | | | | | |
| Asian | 2 | (6) | 5 | (8) | |
| African American | 8 | (2.5) | 6 | (9) | |
| Caucasian | 5 | (16) | 8 | (12) | |
| Hispanic | 11 | (34) | 14 | (21) | |
| >1 Race | 2 | (6) | 1 | (2) | |

TABLE 6
Antidepressant Classes for Medication-Exposed Participants

| Antidepressant Classes for | Within 3 Months | Lifetime |
|---|---|---|
| Medication- | of PET Scanning | Exposure |
| Exposed Participants | (n = 37) | (n = 66) |
| SSRI | 19 | 53 |
| SNRI | 12 | 28 |
| MAOI | 3 | 7 |
| Tricyclic antidepressant | 3 | 8 |
| Melatonergic Agonist | 1 | 0 |
| Atypical Antipsychotic | 5 | 9 |
| Mixed | 2 | 7 |
| NERI | 11 | 30 |
| Unknown | 0 | 2 |

Note:
Some participants had multiple antidepressant medication trials and have been counted more than once. Of the 37 participants who were exposed to antidepressants within three months of PET scanning, 16 participants were on more than one antidepressant medication. All 16 were on antidepressant medications of different classes. Of the 66 participants who had lifetime exposure to antidepressant medication, 47 of those participants had more than one antidepressant medication trial. Forty of those participants had medication trials with different classes of antidepressants. Two of the exposed participants had missing lifetime antidepressant medication name data, therefore these two participants were not classified.

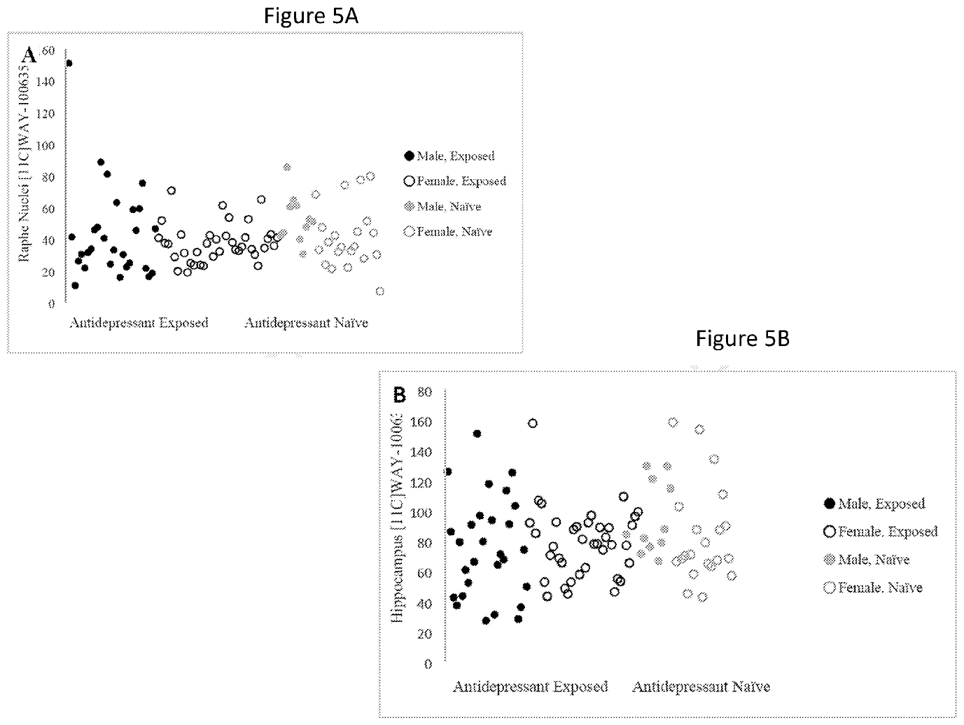

### Example 8—[¹¹C]WAY-100635 BP_{F} in Antidepressant Medication Exposed Vs. Naïve Participants

[¹¹C]WAY-100635 binding across the a priori ROIs did not differ between medication exposed and medication naïve patients after accounting for age and sex (F[1, 95]=2.62, p=0.11). An interaction term for brain region was not significant (F[12, 1152]=0.16, p=1.00), indicating that the relationship between the two groups did not differ across the a priori brain regions. [¹¹C]WAY-100635 BP_{F} in both groups for the raphe nuclei, where 5-HT_{1A} autoreceptors are localized, and hippocampus, a brain region rich in post-synaptic 5-HT_{1A} receptors, are shown in FIG. 5. This result held true for BP_{P} (F[1, 95]=0.003, p=0.95) as well as BP_{ND} (F[1, 95]=3.66, p=0.06). The interaction terms for each of these outcome measures were also not significant (BP_{P} F[12, 1152]=0.29, p=0.99; BP_{ND} F[12, 1152]=0.50, p=0.92).

Similarly, [¹¹C]WAY-100635 binding across the a priori ROIs did not differ between SSRI/SNRI exposed and medication naïve participants after accounting for age and sex (F[1, 86]=3.73, p=0.06). An interaction term for brain region was not significant (F[12, 1044]=0.08, p=1.00), indicating that the relationship between the two groups did not differ across the a priori brain regions. This result held true for BP_{P} (F[1, 86]=0.11, p=0.75) as well as BP_{ND} (F[1, 86]=3.25, p=0.08). The interaction terms for each of these outcome measures were also not significant (BP_{P} F[12, 1044]=0.17, p=1.00; BP_{ND} F[12, 1044]=0.35, p=0.98).

### Example 9—[¹¹C]WAY-100635 BP_{F} and Time Off Antidepressants

There was no main effect of time off any antidepressants on [¹¹C]WAY-100635 binding across all a priori ROIs after accounting for age and sex (F[1, 63]=0.36, p=0.55). The relationships between [¹¹C]WAY-100635 BP_{F} and weeks off antidepressants for the raphe nuclei and hippocampus are shown in FIG. 6. The results were the same when specifying to time off SSRI/SNRIs (F[1, 25]=0.38, p=0.54). These analyses were repeated using BP_{P} and BP_{ND} as outcome measures and similar results were obtained when looking at all antidepressants (BP_{P}, F[1, 63]=3.22, p=0.08; BP_{ND}, F[1, 63]=1.70, p=0.20) and when specifying to SSRI/SSNRIs (BP_{P}, F[1, 25]=0.80, p=0.38; BP_{ND}, F[1, 25]=0.14, p=0.71).

### Example 10—Comparison of Raphe and Hippocampal Binding in Medication Naïve, Medication Exposed but Washed Out and Before and after Medication Treatment (Last Two Groups are from Published Data in Gray et al., 2013)

The results are shown in Table 7. The pretreatment binding levels, BP_{F}, in raphe and hippocampus in Gray et al. (2013) were comparable to the binding levels in the medication naïve and medication washed out MDD groups. The currently SSRI-treated group has lower binding.

TABLE 7
Raphe Nuclei binding for Gray et al. sample and our
medication naive and medication exposed samples

| | BP_{F} | BP_{P} | BP_{ND} |
|---|---|---|---|
| Gray et al. Pre- | 40.04 ± 15.39 | 2.37 ± 0.68 | 8.44 ± 3.74 |
| Treatment (n = 19) | | | |
| Gray et al. Post- | 34.91 ± 12.55 | 2.33 ± 1.07 | 8.27 ± 2.72 |
| Treatment (n = 19) | | | |
| Medication Naïve | 45.18 ± 18.61 | 2.51 ± 0.95 | 9.32 ± 2.82 |
| (n = 32) | | | |
| Medication Exposed | 39.79 ± 21.11 | 2.44 ± 0.98 | 8.53 ± 3.41 |
| (n = 66) | | | |

### REFERENCES

Albert and Lemonde, 2004. 5-HT1A receptors, gene repression, and depression: guilt by association. The Neuroscientist: a review journal bringing neurobiology, neurology and psychiatry 10, 575-593.
Ardekani et al., 2005. Quantitative comparison of algorithms for inter-subject registration of 3D volumetric brain MRI scans. J. Neurosci. Methods 142, 67-76.
Avants et al., 2011. An open source multivariate framework for n-tissue segmentation with evaluation on public data. Neuroinformatics 9, 381-400.
Beck et al., 1961. An inventory for measuring depression. Archives of General Psychiatry 4, 561-571.
Blier et al., 1990. A role for the serotonin system in the mechanism of action of antidepressant treatments: preclinical evidence. The Journal of Clinical Psychiatry 51 Suppl, 14-20; discussion 21.
Carlson et al., 2013. Neural correlates of rapid antidepressant response to ketamine in treatment-resistant unipolar depression: a preliminary positron emission tomography study. Biological Psychiatry 73, 1213-1221.
C C et al., 1996. A Single Scatter Simulation Technique fo Scatter Correction in 3D PET, in: Grangeat, P., Amans, J.-L. (Eds.), Three-dimensional image reconstruction in radiology and nuclear medicine. Kluwer Academic Publishers, Dordrecht; Boston, pp. 255-268.
Compan et al., 1998. Differential effects of serotonin (5-HT) lesions and synthesis blockade on neuropeptide-Y immunoreactivity and 5-HT_{1A}, 5-HT_{1B/1D} and 5-HT_{2A/2C} receptor binding sites in the rat cerebral cortex. Brain Research 795, 264-276.
Czesak et al., 2006. Cell-specific repressor or enhancer activities of Deaf-1 at a serotonin 1A receptor gene polymorphism. J. Neurosci. 26, 1864-1871.
DeLorenzo et al., 2009. A new method for assessing PET-MRI coregistration, SPIE Medical Imaging, Florida, USA, pp. 72592W-72592W-72598.
DeLorenzo et al. 2013. Prediction of selective serotonin reuptake inhibitor response using diffusion-weighted MRI. Frontiers in Psychiatry 4(5).
Dunn and Clark, 1987. Applied statistics: analysis of variance and regression, Wiley series in probability and mathematical statistics Applied probability and statistics, 2nd ed. Wiley, New York, pp. xii, 445 p.
First et al., 1995. Structured Clinical Interview for DSM-IV Axis I Disorders (SCID-UP, Version 2.0). Biometrics Research Dept., New York State Psychiatric Institute, New York.
Golland and Fischl, 2003. Permutation tests for classification: towards statistical significance in image-based studies. Inf. Process Med. Imaging 18, 330-341.
Gottesman and Shields, 1973. Genetic theorizing and schizophrenia. British Journal of Psychiatry. 122:15-30.
Gray et al., 2013. Antidepressant treatment reduces serotonin 1A autoreceptor binding in major depressive disorder. Biological Psychiatry 74, 26-31.
Greenberg et al., 2015. The economic burden of adults with major depressive disorder in the United States (2005 and 2010). J Clin Psychiatry, 76(2), 155-62.
Gunn et al., 2002. Positron emission tomography compartmental models: a basis pursuit strategy for kinetic modeling. Journal of Cerebral Blood Flow and Metabolism 22, 1425-1439.
Hamilton 1960. A rating scale for depression. Journal of Neurology, Neurosurgery, and Psychiatry, 23, 56-62.
Hanley and McNeil, 1982. The meaning and use of the area under a receiver operating characteristic (ROC) curve. Radiology 143, 29-36.
Hesselgrave and Parsey, 2013. Imaging the serotonin 1A receptor using [¹¹C]WAY100635 in healthy controls and major depression. Philosophical transactions of the Royal Society of London. Series B, Biological sciences 368, 20120004.
Hirvonen et al., 2008. Decreased brain serotonin 5-HT1A receptor availability in medication-naïve patients with major depressive disorder: An in-vivo imaging study using PET and [^{carbonyl-11C}]WAY-100635. International Journal of Neuropsychopharmacology 11, 465-476.
Innis et al., 2007. Consensus nomenclature for in vivo imaging of reversibly binding radioligands. Journal of Cerebral Blood Flow and Metabolism 27, 1533-1539.
Kaufman et al., 2015. Quantification of the Serotonin 1A Receptor Using PET: Identification of a Potential Biomarker of Major Depression in Males. Neuropsychopharmacology. 40(7), 1692-99.
Klaassen et al., 1999. Mood effects of 24-hour tryptophan depletion in healthy first-degree relatives of patients with affective disorders. Biological Psychiatry 46, 489-497.
Klein et al., 2009. Evaluation of 14 nonlinear deformation algorithms applied to human brain MRI registration. NeuroImage 46, 786-802.
Leboyer et al., 1998. Psychiatric genetics: search for phenotypes. Trends Neuroscience 21, 102-105.
Lemonde et al., 2003. Impaired repression at a 5-hydroxytryptamine 1A receptor gene polymorphism associated with major depression and suicide. J. Neurosci. 23, 8788-8799.
Mann et al., 2005 Family history of suicidal behavior and mood disorders in probands with mood disorders. The American Journal of Psychiatry 162, 1672-1679.
Mawlawi et al., 2001. Imaging human mesolimbic dopamine transmission with positron emission tomography: I. Accuracy and precision of D(2) receptor parameter measurements in ventral striatum. Journal of Cerebral Blood Flow and Metabolism 21, 1034-1057.
Milak et al. 2010. In vivo quantification of human serotonin 1A receptor using 11C-CUMI-101, an agonist PET radiotracer. Journal of Nuclear Medicine 51, 1892-1900.
Miller et al., 2009. Elevated serotonin 1A binding in remitted major depressive disorder: evidence for a trait biological abnormality. Neuropsychopharmacology 34, 2275-2284.
Miller et al., 2013. Brain serotonin 1A receptor binding as a predictor of treatment outcome in major depressive disorder. Biological Psychiatry 74, 760-767.
NIMH, Major Depression Among Adults. National Institute of Mental Health, 2015.
Ogden and Tarpey, 2006. Estimation in regression models with externally estimated parameters. Biostatistics 7, 115-129.
Oquendo et al., 2003. Risk factors for suicidal behavior: The utility and limitations of research instruments. Standard Evaluation in Clinical Practice, 103-130.
Pantazatos et al., 2012. Decoding unattended fearful faces with whole-brain correlations: an approach to identify condition-dependent large-scale functional connectivity. PLoS Comput Biol 8, e1002441.
Parsey et al., 2005. Regional Heterogeneity of 5-HT1A Receptors in Human Cerebellum as Assessed by Positron Emission Tomograph. Journal of Cerebral Blood Flow and Metabolism 25, 785-93.
Parsey et al., 2010a. Higher serotonin 1A binding in a second major depression cohort: modeling and reference region considerations. Biological Psychiatry 68, 170-178.
Parsey et al., 2010b. Higher Serotonin 1A Binding in a Second Major Depression Cohort: Modeling and Reference Region Considerations. Biological Psychiatry, 68, 170-78.
Parsey et al., 2006. Altered serotonin 1A binding in major depression: a [carbonyl-C-11]WAY100635 positron emission tomography study. Biological Psychiatry 59, 106-113.
Parsey et al., 2001. Comparison of Kinetic Modeling Methods for the In Vivo Quantification of 5-HT1A Receptors Using WAY 100635, in: Gjedde, A., Hansen, S. B., Knudsen, G. M., Paulson, O. B. (Eds.), Physiological Imaging of the Brain with PET. Academic Press, San Diego, pp. 249-255.
Parsey et al., 2000. Validation and reproducibility of measurement of 5-HT1A receptor parameters with [carbonyl-¹¹C]WAY-100635 in humans: comparison of arterial and reference tisssue input functions. Journal of Cerebral Blood Flow and Metabolism 20, 1111-1133.
Peterson and Weissman, 2011. A brain-based endophenotype for major depressive disorder. Annu. Rev. Med. 62, 461-474.
Slifstein et al., 2000. Derivation of [(11)C]WAY-100635 binding parameters with reference tissue models: effect of violations of model assumptions. Nuclear mmedicine and Biology 27, 487-492.
Sullivan et al., 2009. Positron emission tomography quantification of serotonin 1A receptor binding in medication-free bipolar depression. Biological Psychiatry 66, 223-230.
Sullivan et al., 2015. Positron emission tomography quantification of serotonin (1A) receptor binding in suicide attempters with major depressive disorder. JAMA Psychiatry 72, 169-178.
Tauscher et al., 2001. Serotonin 5-HT1A receptor binding potential declines with age as measured by [¹¹C]WAY-100635 and PET. Neuropsychopharmacology 24, 522-530.
Uhl and Grow, 2004. The burden of complex genetics in brain disorders. Archives of General Psychiatry 61, 223-229.
Vapnik, 1999. An overview of statistical learning theory. IEEE transactions on neural networks/a publication of the IEEE Neural Networks Council 10, 988-999.
WHO, Depression. World Health Organization, 2017.
Wu et al., 2007. Optimal metabolite curve fitting for kinetic modeling of ¹¹C-WAY-100635. Journal of Nuclear Medicine 48, 926-931.
Zimmerman, 1994. Interview guide for evaluating DSM-IV psychiatric disorders and the mental status examination. Psych Products Press.

1.-36. (canceled)

37. A method of identifying a subject at risk for or having major depressive disorder, comprising:
a. introducing a radioligand capable of binding a serotonin 1A receptor into the subject;
b. performing one or more scans of the subject;
c. detecting serotonin 1A receptor binding potential in the brain of the subject;
d. comparing the serotonin 1A receptor binding potential in the brain of the subject to a reference value; and
e. determining the subject is at high risk or has major depressive disorder if the serotonin 1A receptor binding potential in the brain of the subject is greater than the reference value.

38. (canceled)

39. (canceled)

40. The method of claim 37, wherein the scans are chosen from the group consisting of positron emission tomography (PET) and single-photon emission computed tomography (SPECT).

41. The method of claim 37, wherein the entire brain is scanned.

42. The method of claim 37, wherein an area of the brain is scanned chosen from the group consisting of the: anterior cingulate; amygdala; cingulate body; dorsolateral prefrontal cortex; hippocampus; insula; medial prefrontal cortex; occipital lobe; orbital prefrontal cortex; parietal lobe; parahippocampal gyrus; temporal lobe; raphe nuclei; and combinations thereof.

43. (canceled)

44. The method of claim 37, wherein the reference value is the serotonin 1A receptor binding potential of a healthy comparison subject or healthy comparison group.

45. The method of claim 37, wherein the reference value ranges from 50 ml/cm³ to about 80 ml/cm³.

46.-48. (canceled)

49. A method of identifying a subject at high risk for or having major depressive disorder, comprising:
a. introducing a radioligand capable of binding a serotonin 1A receptor into the subject;
b. performing one or more scans of the subject;
c. detecting serotonin 1A receptor binding potential pattern in the brain of the subject;
d. comparing the serotonin 1A receptor binding potential pattern in the brain of the subject to a reference serotonin 1A receptor binding potential pattern; and
e. determining that the subject is at high risk or has major depressive disorder if the serotonin 1A receptor binding potential pattern in the brain of the subject is the same or similar to a subject with major depressive disorder.

50. (canceled)

51. (canceled)

52. The method of claim 49, wherein the scans are chosen from the group consisting of positron emission tomography (PET) and single-photon emission computed tomography (SPECT).

53. The method of claim 49, wherein the entire brain is scanned.

54. The method of claim 49, wherein an area of the brain is scanned chosen from the group consisting of the: anterior cingulate; amygdala; cingulate body; dorsolateral prefrontal cortex; hippocampus; insula; medial prefrontal cortex; occipital lobe; orbital prefrontal cortex; parietal lobe; parahippocampal gyrus; temporal lobe; raphe nuclei; and combinations thereof.

55. (canceled)

56. The method of claim 49, wherein the serotonin 1A receptor binding potential pattern is obtained using multivoxel pattern analysis

57. A method of identifying a subject at high risk for or having for major depressive disorder, comprising:
a. introducing a radioligand capable of binding a serotonin 1A receptor into the subject;
b. further introducing a PET tracer;
c. detecting a percent free fraction (f_{P}) of the PET tracer in the subject;
d. comparing the percent free fraction (f_{P}) of the PET tracer in the subject to a reference value of percent free fraction (f_{P}) of the PET tracer; and
e. determining that the subject is at high risk or has major depressive disorder if the percent free fraction (f_{P}) of the PET tracer in the subject is equal or less than the reference value.

58. (canceled)

59. (canceled)

60. The method of claim 53, wherein the reference value is about 6.2%.

61.-71. (canceled)

72. The method of claim 37, further comprising administering to the subject a therapeutically effective amount of therapeutic agent for major depressive disorder and/or treating the subject with psychotherapy, wherein the subject was determined to be at high risk or have major depressive disorder in step e.

73. The method of claim 72, wherein the therapeutic agent is chosen from the group consisting of selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine and monoamine oxidase inhibitors (MAOIs).

74. The method of claim 49, further comprising administering to the subject a therapeutically effective amount of therapeutic agent for major depressive disorder and/or treating the subject with psychotherapy, wherein the subject was determined to be at high risk or have major depressive disorder in step e.

75. The method of claim 74, wherein the therapeutic agent is chosen from the group consisting of selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine and monoamine oxidase inhibitors (MAOIs).

76. The method of claim 57, further comprising administering to the subject a therapeutically effective amount of therapeutic agent for major depressive disorder and/or treating the subject with psychotherapy, wherein the subject was determined to be at high risk or have major depressive disorder in step e.

77. The method of claim 76, wherein the therapeutic agent is chosen from the group consisting of selective serotonin reuptake inhibitors (SSRIs), serotonin and norepinephrine reuptake inhibitors (SNRIs), atypical antidepressants, tricyclic antidepressants, tetracyclic antidepressants, lithium, electroconvulsive therapy, rapid transcranial magnetic stimulation and related stimulation therapies, anticonvulsants, ketamine and monoamine oxidase inhibitors (MAOIs).
